(19) United States
(12) Patent Application Publication
(10) Pub. No.: US 2020/0040404 A1
(43) Pub. Date: Feb. 6, 2020

(54) COMPOSITIONS AND METHODS FOR DETECTING PROSTATE CANCER

(71) Applicant: Cell MdX, LLC, Corvallis, OR (US)

(72) Inventors: Amin I. Kassis, Corvallis, OR (US); Ricardo Henao, Corvallis, OR (US); Kirk Wojno, Corvallis, OR (US); Geoffrey Erickson, Corvallis, OR (US); Harry Stylli, Corvallis, OR (US)

(21) Appl. No.: 16/527,200

(22) Filed: Jul. 31, 2019

Related U.S. Application Data
(60) Provisional application No. 62/826,147, filed on Mar. 29, 2019.
(60) Provisional application No. 62/712,720, filed on Jul. 31, 2018.

(51) Int. Cl. C12Q 1/6886

(52) U.S. Cl. CPC C12Q 1/6886; C12Q 2600/158; C12Q 2600/112

(57) ABSTRACT

The present invention relates to compositions and methods for assessing prostate cancer (e.g., identification of the aggressiveness or indolence of prostate cancer) in a subject. The compositions and methods include obtaining subject specific information (e.g., age, digital rectal exam (DRE) data, prostate volume, total prostate-specific antigen (PSA)) and obtaining a biological sample from a subject and determining a measurement for a panel of biomarkers in the biological sample. Compositions and methods of the invention find use in both clinical and research settings, for example, within the fields of biology, immunology, medicine, and oncology.

### CROSS REFERENCE TO RELATED APPLICATIONS

This application claims priority to and the benefit of U.S. Provisional Application No. 62/826,147, filed Mar. 29, 2019 and U.S. Provisional Application No. 62/712,720, filed Jul. 31, 2018, which are hereby incorporated by reference in their entireties.

### FIELD OF THE INVENTION

The present invention relates to compositions and methods for assessing prostate cancer (e.g., identification of the aggressiveness or indolence of prostate cancer) in a subject. The compositions and methods include obtaining subject specific information (e.g., age, digital rectal exam (DRE) data, prostate volume, total prostate-specific antigen (PSA)) and obtaining a biological sample from a subject and determining a measurement for a panel of biomarkers in the biological sample. Compositions and methods of the invention find use in both clinical and research settings, for example, within the fields of biology, immunology, medicine, and oncology.

### BACKGROUND

Prostate cancer is the second most common type of cancer and the fifth leading cause of cancer-related death in men (World Cancer Report 2014. World Health Organization. 2014). In 2012, it occurred in 1.1 million men and caused 307,000 deaths. It was the most common cancer in males in 84 countries (World Cancer Report 2014. World Health Organization. 2014. pp. Chapter 5.11), occurring more commonly in the developed world where rates of occurrence have been increasing.

Early diagnosis of prostate cancer often increases the likelihood of successful treatment or cure of such disease. Current diagnostic methods, however, depend largely on population-derived average values obtained from healthy individuals.

Personalized diagnostic methods are needed that enable the diagnosis, especially the early diagnosis, of the presence of prostate cancer in individuals who are not known to have the cancer or who have recurrent prostate cancer.

Leukocytes begin as pluripotent hematopoietic stem cells in the bone marrow and develop along either the myeloid lineage (monocytes, macrophages, neutrophils, eosinophils, and basophils) or the lymphoid lineage (T and B lymphocytes and natural killer cells). The major function of the myeloid lineage cells (e.g., neutrophils and macrophages) is the phagocytosis of infectious organisms, live unwanted damaged cells, senescent and dead cells (apoptotic and necrotic), as well as the clearing of cellular debris. Phagocytes from healthy animals do not replicate and are diploid, i.e., have a DNA content of 2n. On average, each cell contains <10 ng DNA, <20 ng RNA, and <300 ng of protein. Non-phagocytic cells are also diploid and are not involved in the internalization of dead cells or infectious organisms and have a DNA index of one.

The lifetime of various white blood cell subpopulations varies from a few days (e.g., neutrophils) to several months (e.g., macrophages). Like other cell types, leukocytes age and eventually die. During their aging process, human blood- and tissue-derived phagocytes (e.g., neutrophils) exhibit all the classic markers of programmed cell death (apoptosis), including caspase activation, pyknotic nuclei, and chromatin fragmentation. These cells also display a number of “eat-me” flags (e.g., phosphatidylserine, sugars) on the extracellular surfaces of their plasma membranes. Consequently, dying and dead cells and subcellular fragments thereof are cleared from tissues and blood by other phagocytic cells.

Although prostate-specific antigen (PSA) is considered an effective tumor marker and generally organ specific, it is not cancer specific. There is considerable overlap in PSA concentrations in men with prostate cancer and men with benign prostatic diseases. PSA does not differentiate men with organ confined prostate cancer (that may benefit from surgery) from those men with non-organ confined prostate cancer (that would not benefit from surgery). Therefore, PSA is not effective in selecting patients for radical prostatectomy.

While PSA is currently one of the most widely used diagnostic measures used to detect prostate cancer, false positives and false negatives are common, resulting in mistreatment of patients with no prostate cancer or overtreatment of patients with non-lethal prostate cancer. Improved methods for detecting prostate cancer are needed.

### SUMMARY

The present invention relates to compositions and methods for assessing prostate cancer (e.g., identification of the aggressiveness or indolence of prostate cancer) in a subject. Compositions and methods of the invention find use in the identification, characterization, and classification (e.g., via computing aggressiveness index) of cancer in a subject.

In some embodiments, the invention provides a method for identifying, assessing and/or predicting the aggressiveness or indolence of cancer (e.g., prostate cancer) in a subject (e.g., a subject suspected of having cancer, a subject diagnosed with a cancer, or a subject at risk for cancer). In some embodiments, the invention provides a method for identifying, assessing and/or predicting the aggressiveness or indolence of prostate cancer (e.g., in a patient previously diagnosed with prostate cancer).

In some embodiments, the invention provides a method of measuring a panel of biomarkers in a subject comprising obtaining a biological sample from the subject; determining a measurement for the panel of biomarkers in the biological sample, wherein the panel of biomarkers comprise one or more (e.g., two, three, four, five, six, seven, eight, nine, ten, eleven, twelve, thirteen, fourteen, fifteen, or more) biomarkers selected from those shown in Table 1, Table 2, and/or Table 3, and wherein the measurement comprises measuring a level of each of the biomarkers in the panel. In some embodiments, the panel of biomarkers comprises one or more (e.g., two, three, four, five, six, seven, eight, nine, ten, eleven, twelve, thirteen, fourteen, fifteen, or more) biomarkers selected from the clinical and genomic covariates shown in Table 1, and/or the genomic covariates listed in Table 2 (e.g., BAMBI, C3orf67, C9orf135, COCH, FLJ40194, FST, FSTL1, GATA2, HDGFRP3, MYO1D, OOEP, SNORD42A, tAKR, TMEM133, WNT9A) and Table 3 (C11orf94, C9orf135, DSP, EGFL6, FST, FSTL1, GATA2, GRID1, KLF17, KRTAP5-8, MID1, MYO1D, OOEP, RSPH9 and TAGLN3). In some embodiments, measuring the panel of biomarkers in the subject identifies, assesses, and/or predicts the aggressiveness or indolence of cancer (e.g., prostate cancer) in a subject (e.g., a subject suspected of having cancer, a subject diagnosed with a cancer, or a subject at risk for cancer). In some embodiments, the biological sample comprises CD2⁺ cells and/or CD14⁺ cells. In one embodiments, determining a measurement for the panel of biomarkers in the biological sample comprises measuring a level of each of the biomarkers in the panel in CD2⁺ cells and/or CD14⁺ cells. In a one embodiment, the method further comprises obtaining one or more clinical data from the subject selected from the group consisting of age, race, digital rectal exam (DRE), prostate volume, and total prostate-specific antigen (PSA). The invention is not limited by the type of clinical data obtained and/or used. Additional examples of clinical data include, but are not limited to, tumor stage, tumor grade, tumor size, tumor visual characteristics, tumor growth, tumor thickness, tumor progression, tumor metastasis, tumor distribution within the body, odor, molecular pathology, genomics, and/or tumor angiograms. In some embodiments, the one or more clinical data are used as clinical covariates and concatenated with the biomarker levels and input into a sparse rank regression model/algorithm (e.g., in order to identify, assess, and/or predict the aggressiveness or indolence of cancer (e.g., prostate cancer) in a subject). In one embodiment, the algorithm provides a cancer (e.g., prostate cancer) aggressiveness index value (e.g., 0, 1, 2, 3, or 4) that identifies and characterizes cancer in a subject (e.g., scaled such that a value of 0 characterizes the absence of cancer in the subject ranging to a value of 4 that characterizes the presence of highly aggressive cancer in the subject). In some embodiments, measuring a level of each of the biomarkers in the panel comprises measuring gene expression levels. The invention is not limited by how gene expression levels are measured. Indeed, any means of measuring gene expression levels may be used including, but not limited to, polymerase chain reaction (PCR) analysis, sequencing analysis, electrophoretic analysis, restriction fragment length polymorphism (RFLP) analysis, Northern blot analysis, quantitative PCR, reverse-transcriptase-PCR analysis (RT-PCR), allele-specific oligonucleotide hybridization analysis, comparative genomic hybridization, heteroduplex mobility assay (HMA), single strand conformational polymorphism (SSCP), denaturing gradient gel electrophoresis (DGGE), RNAase mismatch analysis, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), surface plasmon resonance, Southern blot analysis, in situ hybridization, fluorescence in situ hybridization (FISH), chromogenic in situ hybridization (CISH), immunohistochemistry (IHC), microarray, comparative genomic hybridization, karyotyping, multiplex ligation-dependent probe amplification (MLPA), Quantitative Multiplex PCR of Short Fluorescent Fragments (QMPSF), microscopy, methylation specific PCR (MSP) assay, HpaII tiny fragment Enrichment by Ligation-mediated PCR (HELP) assay, radioactive acetate labeling assays, colorimetric DNA acetylation assay, chromatin immunoprecipitation combined with microarray (ChIP-on-chip) assay, restriction landmark genomic scanning, Methylated DNA immunoprecipitation (MeDIP), molecular break light assay for DNA adenine methyltransferase activity, chromatographic separation, methylation-sensitive restriction enzyme analysis, bisulfate-driven conversion of non-methylated cytosine to uracil, methyl-binding PCR analysis, or a combination thereof. In some embodiments, gene expression levels are measured by a sequencing technique such as, but not limited to, direct sequencing, RNA sequencing, whole transcriptome shotgun sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, mass spectrometry, and a combination thereof. In some embodiments, measuring a level of each of the biomarkers in the panel comprises measuring protein expression levels. The invention is not limited to any particular method of measuring protein expression levels. Exemplary methods of measuring protein expression levels include, but are not limited to, an immunohistochemistry assay, an enzyme-linked immunosorbent assay (ELISA), in situ hybridization, chromatography, liquid chromatography, size exclusion chromatography, high performance liquid chromatography (HPLC), gas chromatography, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), radioimmunoassays, microscopy, microfluidic chip-based assays, surface plasmon resonance, sequencing, Western blotting assay, or a combination thereof. In some embodiments, measuring a level of each of the biomarkers in the panel comprises measuring by a qualitative assay, a quantitative assay, or a combination thereof. Exemplary quantitative assays include, but are not limited to, sequencing, direct sequencing, RNA sequencing, whole transcriptome shotgun sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), polymerase chain reaction (PCR) analysis, quantitative PCR, real-time PCR, fluorescence assay, colorimetric assay, chemiluminescent assay, or a combination thereof. In some embodiments, the subject is a human.

In another aspect, the invention provides methods for detecting or diagnosing prostate cancer by using at least one or more (e.g., two, three, four, five, six, seven, eight, nine, ten, eleven, twelve, thirteen, fourteen, fifteen or more) markers selected from Table 1, Table 2, and/or Table 3. Levels (e.g., gene expression levels, protein expression levels, or activity levels) of the selected markers may be measured from phagocytic cells (e.g., macrophages, monocytes, dendritic cells, and/or neutrophils) and from non-phagocytic cells (e.g., T cells), from a subject. Such levels then can be compared, e.g., the levels of the selected markers in the phagocytic cells and in the non-phagocytic cells to identify one or more differences between the measured levels, indicating whether the subject has prostate cancer. The identified difference(s) can also be used for assessing the risk of developing prostate cancer, prognosing prostate cancer, monitoring prostate cancer progression or regression, assessing the efficacy of a treatment for prostate cancer, or identifying a compound capable of ameliorating or treating prostate cancer.

In yet another aspect, the levels of the selected markers in the phagocytic cells may be compared to the levels of the selected markers in a control (e.g., a normal or healthy control subject, or a normal or healthy cell from the subject) to identify one or more differences between the measured levels, indicating whether the subject has prostate cancer, the prognosis of the cancer and the monitoring of the cancer. The identified difference(s) can also be used for assessing the risk of developing prostate cancer, prognosing prostate cancer, monitoring prostate cancer progression or regression, assessing the efficacy of a treatment for prostate cancer, or identifying a compound capable of ameliorating or treating prostate cancer.

In some embodiments, the invention provides a method for diagnosing or aiding in the diagnosis of prostate cancer in a subject, the method comprising the steps of:

a) measuring the levels of one or more (e.g., two, three, four, five, six, seven, eight, nine, ten, eleven, twelve, thirteen, fourteen, fifteen or more) markers selected from Table 1, Table 2, and/or

Table 3 in a population of the subject's macrophage or monocyte cells;

b) measuring the levels of the one or more selected markers in a population of the subject's non-phagocytic cells; and

c) identifying a difference between the measured levels of the one or more selected markers in steps a) and b), wherein the identified difference indicates that the subject has said prostate cancer.

In some embodiments, the invention provides a method for diagnosing or aiding in the diagnosis of prostate cancer in a subject, the method comprising the steps of:

a) measuring the levels of one or more markers selected from BAMBI, C3orf67, C9orf135, C11orf94, COCH, DSP, EGFL6, FLJ40194, FST, FSTL1, GATA2, GRID1, HDGFRP3, KLF17, KRTAP5-8, MID1, MYO1D, OOEP, RSPH9, SNORD42A, TAGLN3, tAKR, TMEM133, and WNT9A in a population of the subject's macrophage or monocyte cells;

b) measuring the levels of the one or more markers selected from BAMBI, C3orf67, C9orf135, C11orf94, COCH, DSP, EGFL6, FLJ40194, FST, FSTL1, GATA2, GRID1, HDGFRP3, KLF17, KRTAP5-8, MID1, MYO1D, OOEP, RSPH9, SNORD42A, TAGLN3, tAKR, TMEM133, and WNT9A in a population of the subject's non-phagocytic cells; and

c) identifying a difference between the measured levels of the one or more selected markers in steps a) and b), wherein the identified difference indicates that the subject has said prostate cancer.

In other embodiments, the invention provides a method for assessing the risk of developing prostate cancer in a subject, the method comprising the steps of:

a) measuring the levels of one or more (e.g., two, three, four, five, six, seven, eight, nine, ten, eleven, twelve, thirteen, fourteen, fifteen or more) markers selected from Table 1, Table 2, and/or Table 3. in a population of the subject's macrophage or monocyte cells;

b) measuring the levels of the one or more selected markers in a population of the subject's non-phagocytic cells; and

c) identifying a difference between the measured levels of the one or more selected markers in steps a) and b), wherein the identified difference indicates that the subject has said prostate cancer.

In other embodiments, the invention provides a method for assessing the risk of developing prostate cancer in a subject, the method comprising the steps of:

a) measuring the levels of one or more markers selected from BAMBI, C3orf67, C9orf135, C11orf94, COCH, DSP, EGFL6, FLJ40194, FST, FSTL1, GATA2, GRID1, HDGFRP3, KLF17, KRTAP5-8, MID1, MYO1D, OOEP, RSPH9, SNORD42A, TAGLN3, tAKR, TMEM133, and WNT9A in a population of the subject's macrophage or monocyte cells;

b) measuring the levels of the one or more markers selected from BAMBI, C3orf67, C9orf135, C11orf94, COCH, DSP, EGFL6, FLJ40194, FST, FSTL1, GATA2, GRID1, HDGFRP3, KLF17, KRTAP5-8, MID1, MYO1D, OOEP, RSPH9, SNORD42A, TAGLN3, tAKR, TMEM133, and WNT9A in a population of the subject's non-phagocytic cells; and

c) identifying a difference between the measured levels of the one or more selected markers in steps a) and b), wherein the identified difference indicates that the subject has said prostate cancer.

In some embodiments, the invention provides a method for prognosing or aiding in the prognosis of prostate cancer in a subject, the method comprising the steps of:

a) measuring the levels of one or more (e.g., two, three, four, five, six, seven, eight, nine, ten, eleven, twelve, thirteen, fourteen, fifteen or more) markers selected from Table 1, Table 2, and/or Table 3 in a population of the subject's macrophage or monocyte cells;

b) measuring the levels of the one or more selected markers in a population of the subject's non-phagocytic cells; and

c) identifying a difference between the measured levels of the one or more selected markers in steps a) and b), wherein the identified difference indicates that the subject has said prostate cancer.

In some embodiments, the invention provides a method for prognosing or aiding in the prognosis of prostate cancer in a subject, the method comprising the steps of:

a) measuring the levels of one or more markers selected from BAMBI, C3orf67, C9orf135, C11orf94, COCH, DSP, EGFL6, FLJ40194, FST, FSTL1, GATA2, GRID1, HDGFRP3, KLF17, KRTAP5-8, MID1, MYO1D, OOEP, RSPH9, SNORD42A, TAGLN3, tAKR, TMEM133, and WNT9A in a population of the subject's macrophage or monocyte cells;

b) measuring the levels of the one or more markers selected from BAMBI, C3orf67, C9orf135, C11orf94, COCH, DSP, EGFL6, FLJ40194, FST, FSTL1, GATA2, GRID1, HDGFRP3, KLF17, KRTAP5-8, MID1, MYO1D, OOEP, RSPH9, SNORD42A, TAGLN3, tAKR, TMEM133, and WNT9A in a population of the subject's non-phagocytic cells; and

c) identifying a difference between the measured levels of the one or more selected markers in steps a) and b), wherein the identified difference indicates that the subject has said prostate cancer.

In some embodiments, the invention provides a method for assessing the efficacy of a treatment for prostate cancer in a subject comprising:

a) measuring the levels of one or more (e.g., two, three, four, five, six, seven, eight, nine, ten, eleven, twelve, thirteen, fourteen, fifteen or more) markers selected from Table 1, Table 2, and/or Table 3 in a population of the subject's macrophage or monocyte cells before the treatment;

b) measuring the levels of the one or more selected markers in a population of the subject's non-phagocytic cells before the treatment;

c) identifying a first difference between the measured levels of the one or more selected markers in steps a) and b);

d) measuring the levels of the one or more selected markers in a population of the subject's macrophage or monocyte cells after the treatment;

e) measuring the levels of the one or more selected markers in a population of the subject's non-phagocytic cells after the treatment;

f) identifying a second difference between the measured levels of the one or more selected markers in steps d) and e); and

g) identifying a difference between the first difference and the second difference, wherein the difference identified in g) is indicative of the efficacy of the treatment for said prostate cancer in the subject.

In some embodiments, the invention provides a method for assessing the efficacy of a treatment for prostate cancer in a subject comprising:

a) measuring the levels of one or more markers selected BAMBI, C3orf67, C9orf135, C11orf94, COCH, DSP, EGFL6, FLJ40194, FST, FSTL1, GATA2, GRID1, HDGFRP3, KLF17, KRTAP5-8, MID1, MYO1D, OOEP, RSPH9, SNORD42A, TAGLN3, tAKR, TMEM133, and WNT9A in a population of the subject's macrophage or monocyte cells before the treatment;

b) measuring the levels of the one or more markers selected from BAMBI, C3orf67, C9orf135, C11orf94, COCH, DSP, EGFL6, FLJ40194, FST, FSTL1, GATA2, GRID1, HDGFRP3, KLF17, KRTAP5-8, MID1, MYO1D, OOEP, RSPH9, SNORD42A, TAGLN3, tAKR, TMEM133, and WNT9A in a population of the subject's non-phagocytic cells before the treatment;

c) identifying a first difference between the measured levels of the one or more selected markers in steps a) and b);

d) measuring the levels of the one or more selected markers in a population of the subject's macrophage or monocyte cells after the treatment;

e) measuring the levels of the one or more selected markers in a population of the subject's non-phagocytic cells after the treatment;

f) identifying a second difference between the measured levels of the one or more selected markers in steps d) and e); and

g) identifying a difference between the first difference and the second difference, wherein the difference identified in g) is indicative of the efficacy of the treatment for said prostate cancer in the subject.

In other embodiments, the invention provides a method for monitoring the progression or regression of prostate cancer in a subject comprising:

a) measuring the levels of one or more (e.g., two, three, four, five, six, seven, eight, nine, ten, eleven, twelve, thirteen, fourteen, fifteen or more) markers selected from Table 1, Table 2, and/or Table 3 in a population of the subject's macrophage or monocyte cells at a first time point;

b) measuring the levels of the one or more selected markers in a population of the subject's non-phagocytic cells at the first time point;

c) identifying a first difference between the measured levels of the one or more selected markers in steps a) and b);

d) measuring the levels of the one or more selected markers in a population of the subject's macrophage or monocyte cells at a second time point;

e) measuring the levels of the one or more selected markers in a population of the subject's non-phagocytic cells at the second time point;

f) identifying a second difference between the measured levels of the one or more selected markers in steps d) and e); and

g) identifying a difference between the first difference and the second difference, wherein the difference identified in g) is indicative of the progression or regression of said prostate cancer in the subject.

In other embodiments, the invention provides a method for monitoring the progression or regression of prostate cancer in a subject comprising:

a) measuring the levels of one or more markers selected from Table 1, Table 2, and/or Table 3 (e.g., BAMBI, C3orf67, C9orf135, C11orf94, COCH, DSP, EGFL6, FLJ40194, FST, FSTL1, GATA2, GRID1, HDGFRP3, KLF17, KRTAP5-8, MID1, MYO1D, OOEP, RSPH9, SNORD42A, TAGLN3, tAKR, TMEM133, and WNT9A) in a population of the subject's macrophage or monocyte cells at a first time point;

b) measuring the levels of the one or more selected markers in a population of the subject's non-phagocytic cells at the first time point;

c) identifying a first difference between the measured levels of the one or more selected markers in steps a) and b);

d) measuring the levels of the one or more selected markers in a population of the subject's macrophage or monocyte cells at a second time point;

e) measuring the levels of the one or more selected markers in a population of the subject's non-phagocytic cells at the second time point;

f) identifying a second difference between the measured levels of the one or more selected markers in steps d) and e); and

g) identifying a difference between the first difference and the second difference, wherein the difference identified in g) is indicative of the progression or regression of said prostate cancer in the subject.

In other embodiments, the invention provides a method for identifying a compound capable of ameliorating or treating prostate cancer in a subject comprising:

a) measuring the levels of one or more (e.g., two, three, four, five, six, seven, eight, nine, ten, eleven, twelve, thirteen, fourteen, fifteen or more) markers selected from Table 1, Table 2, and/or Table 3 in a population of the subject's macrophage or monocyte cells before administering the compound to the subject;

b) measuring the levels of the one or more selected markers in a population of the subject's non-phagocytic cells before administering the compound to the subject;

c) identifying a first difference between the measured levels of the one or more selected markers in steps a) and b);

d) measuring the levels of the one or more selected markers in a population of the subject's macrophage or monocyte cells after the administration of the compound;

e) measuring the levels of the one or more selected markers in a population of the subject's non-phagocytic cells after the administration of the compound;

f) identifying a second difference between the measured levels of the one or more selected markers in steps d) and e); and

g) identifying a difference between the first difference and the second difference, wherein the difference identified in g) indicates that the compound is capable of ameliorating or treating said prostate cancer in the subject.

In other embodiments, the invention provides a method for identifying a compound capable of ameliorating or treating prostate cancer in a subject comprising:

a) measuring the levels of one or more markers selected from BAMBI, C3orf67, C9orf135, C11orf94, COCH, DSP, EGFL6, FLJ40194, FST, FSTL1, GATA2, GRID1, HDGFRP3, KLF17, KRTAP5-8, MID1, MYO1D, OOEP, RSPH9, SNORD42A, TAGLN3, tAKR, TMEM133, and WNT9A in a population of the subject's macrophage or monocyte cells before administering the compound to the subject;

b) measuring the levels of the one or more selected markers in a population of the subject's non-phagocytic cells before administering the compound to the subject;

c) identifying a first difference between the measured levels of the one or more selected markers in steps a) and b);

d) measuring the levels of the one or more selected markers in a population of the subject's macrophage or monocyte cells after the administration of the compound;

e) measuring the levels of the one or more selected markers in a population of the subject's non-phagocytic cells after the administration of the compound;

f) identifying a second difference between the measured levels of the one or more selected markers in steps d) and e); and

g) identifying a difference between the first difference and the second difference, wherein the difference identified in g) indicates that the compound is capable of ameliorating or treating said prostate cancer in the subject.

In some embodiments, the selected markers are measured from the same population of non-phagocytic cells in steps b) or e). In some embodiments, the selected markers are measured from the different populations of non-phagocytic cells in steps b) or e). In some embodiments, at least two, three, four, five, six, seven, eight, nine, ten, eleven, twelve, thirteen, fourteen, fifteen or more markers are selected. The selected markers may be up-regulated or activated in the macrophage, monocyte, and/or neutrophil cells compared to the non-phagocytic cells, or, the selected markers may be down-regulated or inhibited in the macrophage, monocyte, and/or neutrophil cells compared to the non-phagocytic cells. In some embodiments, the methods comprise lysing the macrophage, monocyte, and/or neutrophil cells and the non-phagocytic cells before step a). In some embodiments, the methods comprise extracting the cellular contents from the macrophage, monocyte, and/or neutrophil cells and the non-phagocytic cells before step a). In some embodiments, the non-phagocytic cells are T cells, B cells, null cells, basophils, or mixtures thereof. In some embodiments, the macrophage, monocyte, and/or neutrophil cells are isolated from a bodily fluid sample, tissues, or cells of the subject. In other embodiments, the non-phagocytic cells are isolated from a bodily fluid sample, tissues, or cells of the subject. The invention is not limited by the type of bodily fluid sample. Indeed, multiple types of bodily fluid samples may be used including, but not limited to, blood, urine, stool, saliva, lymph fluid, cerebrospinal fluid, synovial fluid, cystic fluid, ascites, pleural effusion, fluid obtained from a pregnant woman in the first trimester, fluid obtained from a pregnant woman in the second trimester, fluid obtained from a pregnant woman in the third trimester, maternal blood, amniotic fluid, chorionic villus sample, fluid from a preimplantation embryo, maternal urine, maternal saliva, placental sample, fetal blood, lavage and cervical vaginal fluid, interstitial fluid, or ocular fluid. In some embodiments, the measured levels are gene expression levels. The invention is not limited by how the gene expression levels are measured. Indeed, any means of measuring gene expression levels described herein may be used. In some embodiments, the measured levels are protein expression levels. The present invention is also not limited by how protein expression levels are measured. A variety of non-limiting examples of how protein expression levels are measured are described herein. In some embodiments, the levels or activities are measured by a qualitative assay, a quantitative assay, or a combination thereof. Non-limiting examples of quantitative assays include sequencing, direct sequencing, RNA sequencing, whole transcriptome shotgun sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), polymerase chain reaction (PCR) analysis, quantitative PCR, real-time PCR, fluorescence assay, colorimetric assay, chemiluminescent assay, or a combination thereof.

In some embodiments, the invention provides kits for measuring the levels of at least one or more (e.g., two, three, four, five, six, seven, eight, nine, ten, eleven, twelve, thirteen, fourteen, fifteen or more) markers selected from Table 1, Table 2, and/or Table 3, comprising reagents for specifically measuring the levels of the one or more selected markers. The invention is not limited by how the markers are measured. In some embodiments, the reagents comprise one or more antibodies or fragments thereof, oligonucleotides, or aptamers.

In some embodiments, the invention provides kits for measuring the levels of at least one or more markers selected from BAMBI, C3orf67, C9orf135, C11 orf94, COCH, DSP, EGFL6, FLJ40194, FST, FSTL1, GATA2, GRID1, HDGFRP3, KLF17, KRTAP5-8, MID1, MYO1D, OOEP, RSPH9, SNORD42A, TAGLN3, tAKR, TMEM133, and WNT9A, comprising reagents for specifically measuring the levels of the one or more selected markers. The invention is not limited by how the markers are measured. In some embodiments, the reagents comprise one or more antibodies or fragments thereof, oligonucleotides, or aptamers.

These and other embodiments of the subject invention will readily occur to those of skill in the art in view of the disclosure herein.

### DESCRIPTION OF THE DRAWINGS

FIG. 1 depicts a table showing the performance characteristics and receiver operating characteristic (ROC) curves for the discovery 713/1018 and validation 305/1018 sets of patients analyzed and assessed during development of embodiments of the SNEP invention.

FIG. 2 shows a comparison of the ROC curves between SNEP vs. PSA vs. prostate volume.

FIG. 3 shows a table summarizing data from an independent, prospectively enrolled, cohort of N=470 new subjects used to validate the findings of the discovery of the signatures identified in Example 1. Due to differences in the composition of the cohort in terms aggressiveness index proportions (prevalence) relative to the discovery cohort, a matched subset of N=372 subjects matched by aggressiveness index was down-selected from the complete N=470 subject cohort (See FIG. 3).

FIG. 4 shows data regarding a prostate cancer signature in one embodiment of the SNEP invention generated using a total of nineteen covariates: four clinical covariates (age, prostate volume, digital rectal exam (DRE), and PSA) and fifteen transcript biomarkers of Table 3.

FIG. 5A and FIG. 5B shows patient scoring on the prostate cancer aggressiveness index according to one embodiment of the SNEP invention using FIG. 5A) nineteen covariates shown in Table 3, or FIG. 5B) using the same covariates minus DRE.

FIG. 6A and FIG. 6B shows patient scoring on the prostate cancer aggressiveness index according to one embodiment of the SNEP invention compared to Gleason scoring using FIG. 6A) nineteen covariates shown in Table 3, or FIG. 6B) using the same covariates minus DRE.

FIG. 7 is a table showing the Aggressiveness Index (AI) parameters.

FIG. 8 is a schematic representation of the Aggressiveness Index classification system for patients with prostate cancer.

FIGS. 9A-9E are graphs illustrating gene expression signature selection. The number of transcripts (x axis) that maximize the association between the gene expression signature and each of the summaries of the biopsy, namely, Gleason group (FIG. 9A), cores positive (FIG. 9B), maximum involvement (FIG. 9C), aggregated biopsy features (FIG. 9D), and biopsy (FIG. 9E) were selected and the association was quantified via Kendall's τ-b (transcripts are ordered via raw p-values from univariate testing).

FIG. 10 is a graph showing that the SNEP assay's ability to identify aggressive prostate cancer increases as a function of risk of aggressive cancer.

FIG. 11 is a table showing gene expression signature characteristics for clinical and log-transformed genomic expression (CD2/CD14 Ratio) covariates FIG. 12 is a ROC Curve (1,018 pts using weighted sum of covariates to compute each ROC curve (binary comparison)).

FIG. 13 is a table summarizing the ROC curves per SNEP assay for the signature discovery-validation studies in which patients were bled into (a) purple top tubes and the samples processed 4 hours post blood draw, or (b) proprietary OCM tubes and the samples processed 72 hours post blood draw.

FIG. 14 is a schematic diagram illustrating the subtraction-normalized expression of phagocytes (SNEP) assay methodology.

FIGS. 15A-15E are graphs illustrating gene expression signatures for Gleason group (τ-b 0.427p 1.3×10⁻²⁵, m 136) (FIG. 15A), positive cores (τ-b 0.275 p 3.3×10⁻¹¹ m 104) (FIG. 15B), maximum involvement (τ-b 0.564, p 8.5×10⁻⁴⁴, m 174) (FIG. 15C), aggregated biopsy features (τ-b 0.517, p 7.2×10⁻³⁷, m 181) (FIG. 15D), and negative vs. positive biopsy (FIG. 15E). m log transformed differentially expressed transcripts were averaged while accounting for the directionality of change (log fold change sign).

FIG. 16 is a Venn diagram illustrating signature overlap between Gleason Group (m=136), Cores Positive (CP, m=104), Maximum Involvement (MI, m=174), Aggregated Biopsy features (ABF, m=181), and biopsy result (m=196).

FIG. 17 is a graph of reads distribution during RNA sequencing. Median reads per CD2 and CD14 samples were 29.8±7.53 and 33.9±7.45 million reads per sample, respectively.

FIGS. 18A and 18B are graphs showing the distribution of RNA sequencing reads per sample before (FIG. 18A) and after (FIG. 18B) normalization for CD2 samples. FIGS. 18C and 18D are graphs showing the distribution of RNA sequencing reads per sample before (FIG. 18C) and after (FIG. 18D) normalization for CD14 samples. Sample normalization was performed using Trimmed Mean M-value (TMM) normalization.

FIGS. 19A and 19B are graphs illustrating Principal Component Analysis (PCA) projection of log(CD2/CD14) onto the first two principal components (PC1 and PC2, 30.93% and 5.4% variance explained, respectively) before (FIG. 19A) and after (FIG. 19B) removing outlying subjects (circled in FIG. 19A). Subjects (dots) are colored by aggregated biopsy features.

FIGS. 20A-20C are graphs illustrating gene expression signatures for the complete range of Gleason group (τ-b=0.197, p=2.7×10⁻¹², m=100) (FIG. 20A), positive cores (τ-b 0.130, p=1.6×10⁻⁶, m=46) (FIG. 20B), maximum involvement (τ-b=0.333, p=3.4×10⁻³³, m=184) (FIG. 20C). m log transformed differentially expressed transcripts were averaged while accounting for the directionality of change (log fold change sign). In the x-axes, 0 represents negative biopsies, and 7.5 in the Gleason Group panel represents a 4+3 pattern. Significance of adjacent group differences were quantified via Student's t tests.

FIGS. 21A-21E are Venn diagrams which illustrate overlap between gene expression signatures: Gleason group (FIG. 21A), cores positive (FIG. 21B), maximum involvement (FIG. 21C), aggregated biopsy features (FIG. 21D), and overall biopsy result (FIG. 21E).

FIG. 22 shows a list of 54 markers and 6 clinical covariates of identified in prostate cancer patients when a Sparse Rank Regression Model was run 25 times (10-fold cross-validation on subsets of 1,018 patients). The numbers in parenthesis indicate the number of times a transcript/clinical variable showed up in the 25 runs (minimum: 3 times; maximum: 25 times).

FIG. 23 provides a listing of PC covariates (including National Center For Biotechnology Information (NCBI) accession numbers and gene ID numbers available via the internet from the National Center For Biotechnology Information) that may be measured in accordance with the present disclosure.

### DEFINITIONS

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +/−20% or +/−10%, more preferably +/−5%, even more preferably +/−1%, and still more preferably +/−0.1% from the specified value, as such variations are appropriate to perform the disclosed methods.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

As used herein, the terms “biomarker” or “marker” or “biological marker” refer to an analyte (e.g., a nucleic acid, DNA, RNA, peptide, protein, or metabolite) that can be objectively measured and evaluated as an indicator for a biological process. In some embodiments, a marker is differentially detectable in phagocytes and is indicative of the presence or absence of prostate cancer. An analyte is differentially detectable if it can be distinguished quantitatively or qualitatively in phagocytes compared to a control, e.g., a normal or healthy control or non-phagocytic cells.

The terms “sample” or “biological sample” as used herein, refers to a sample of biological fluid, tissue, or cells, in a healthy and/or pathological state obtained from a subject. Such samples include, but are not limited to, blood, bronchial lavage fluid, sputum, saliva, urine, amniotic fluid, lymph fluid, tissue or fine needle biopsy samples, peritoneal fluid, cerebrospinal fluid, nipple aspirates, and includes supernatant from cell lysates, lysed cells, cellular extracts, and nuclear extracts.

The terms “patient,” “subject,” “individual,” and the like are used interchangeably herein and refer to either a human or a non-human animal. These terms include mammals, such as humans, primates, livestock animals (e.g., bovines, porcines), companion animals (e.g., canines, felines) and rodents (e.g., mice and rats).

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors for developing cancer. A subject suspected of having cancer has generally not been tested for cancer. However, a “subject suspected of having cancer” encompasses an individual who has received a preliminary diagnosis (e.g., a CT scan showing a mass) but for whom a confirmatory test (e.g., biopsy and/or histology) has not been done or for whom the type and/or stage of cancer is not known. The term further includes people who previously had cancer (e.g., an individual in remission). A “subject suspected of having cancer” is sometimes diagnosed with cancer and is sometimes found to not have cancer.

As used herein, the term “subject diagnosed with a cancer” refers to a subject who has been tested and found to have cancerous cells. The cancer may be diagnosed using any suitable method, including but not limited to, biopsy, x-ray, blood test, etc.

As used herein, the term “subject at risk for cancer” refers to a subject with one or more risk factors for developing a specific cancer. Risk factors include, but are not limited to, gender, age, genetic predisposition, environmental exposure, and previous incidents of cancer, preexisting non-cancer diseases, and lifestyle.

As used herein, the term “characterizing cancer in a subject” refers to the identification of one or more properties of a cancer sample in a subject, including but not limited to, the presence of benign, pre-cancerous or cancerous tissue and the stage of the cancer. In one non-limiting example, compositions and methods of the invention are utilized to characterize cancer in a subject (e.g., to identify the aggressiveness or indolence of prostate cancer) in a subject.

As used herein, the terms “normal control”, “healthy control”, and “not-diseased cells” likewise mean a sample (e.g., cells, serum, tissue) taken from a source (e.g., subject, control subject, cell line) that does not have the condition or disease being assayed and therefore may be used to determine the baseline for the condition or disorder being measured. A control subject refers to any individual that has not been diagnosed as having the disease or condition being assayed. It is also understood that the control subject, normal control, and healthy control, include data obtained and used as a standard, i.e. it can be used over and over again for multiple different subjects. In other words, for example, when comparing a subject sample to a control sample, the data from the control sample could have been obtained in a different set of experiments, for example, it could be an average obtained from a number of healthy subjects and not actually obtained at the time the data for the subject was obtained.

The term “diagnosis” as used herein refers to methods by which the skilled artisan can estimate and/or determine whether or not a patient is suffering from a given disease or condition. In some embodiments, the term “diagnosis” also refers to staging (e.g., Stage I, II, III, or IV) of cancer. The skilled artisan often makes a diagnosis on the basis of one or more diagnostic indicators, e.g., a marker, the presence, absence, amount, or change in amount of which is indicative of the presence, severity, or absence of the condition.

The term “prognosis” as used herein refers to is used herein to refer to the likelihood of prostate cancer progression, including recurrence of prostate cancer.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The “area under curve” or “AUC” refers to area under a ROC curve. AUC under a ROC curve is a measure of accuracy. An AUC of 1 represents a perfect test, whereas an AUC of 0.5 represents an insignificant test. A preferred AUC may be at least approximately 0.700, at least approximately 0.750, at least approximately 0.800, at least approximately 0.850, at least approximately 0.900, at least approximately 0.910, at least approximately 0.920, at least approximately 0.930, at least approximately 0.940, at least approximately 0.950, at least approximately 0.960, at least approximately 0.970, at least approximately 0.980, at least approximately 0.990, or at least approximately 0.995.

“Isolated polynucleotide” as used herein may mean a polynucleotide (e.g., of genomic, cDNA, or synthetic origin, or a combination thereof) that, by virtue of its origin, the isolated polynucleotide is not associated with all or a portion of a polynucleotide with which the “isolated polynucleotide” is found in nature; is operably linked to a polynucleotide that it is not linked to in nature; or does not occur in nature as part of a larger sequence.

A “receiver operating characteristic” curve or “ROC” curve refers to a graphical plot that illustrates the performance of a binary classifier system as its discrimination threshold is varied. For example, an ROC curve can be a plot of the true positive rate against the false positive rate for the different possible cutoff points of a diagnostic test. It is created by plotting the fraction of true positives out of the positives (TPR=true positive rate) vs. the fraction of false positives out of the negatives (FPR=false positive rate), at various threshold settings. TPR is also known as sensitivity, and FPR is one minus the specificity or true negative rate. The ROC curve demonstrates the tradeoff between sensitivity and specificity (any increase in sensitivity will be accompanied by a decrease in specificity); the closer the curve follows the left-hand border and then the top border of the ROC space, the more accurate the test; the closer the curve comes to the 45-degree diagonal of the ROC space, the less accurate the test; the slope of the tangent line at a cutoff point gives the likelihood ratio (LR) for that value of the test; and the area under the curve is a measure of test accuracy.

A variety of cell types, tissue, or bodily fluid may be utilized to obtain a sample. Such cell types, tissues, and fluid may include sections of tissues such as biopsy and autopsy samples, frozen sections taken for histologic purposes, blood (such as whole blood), plasma, serum, red blood cells, platelets, interstitial fluid, cerebral spinal fluid, etc. Cell types and tissues may also include lymph fluid, cerebrospinal fluid, a fluid collected by A tissue or cell type may be provided by removing a sample of cells from a human and a non-human animal, but can also be accomplished by using previously isolated cells (e.g., isolated by another person, at another time, and/or for another purpose). Archival tissues, such as those having treatment or outcome history, may also be used.

“Sensitivity” of an assay as used herein refers to the proportion of subjects for whom the outcome is positive that are correctly identified as positive.

“Specificity” of an assay as used herein refers to the proportion of subjects for whom the outcome is negative that are correctly identified as negative.

“Solid phase” or “solid support” as used interchangeably herein, refers to any material that can be used to attach and/or attract and immobilize (1) one or more capture agents or capture specific binding partners, or (2) one or more detection agents or detection specific binding partners. The solid phase can be chosen for its intrinsic ability to attract and immobilize a capture agent. Alternatively, the solid phase can have affixed thereto a linking agent that has the ability to attract and immobilize the (1) capture agent or capture specific binding partner, or (2) detection agent or detection specific binding partner. For example, the linking agent can include a charged substance that is oppositely charged with respect to the capture agent (e.g., capture specific binding partner) or detection agent (e.g., detection specific binding partner) itself or to a charged substance conjugated to the (1) capture agent or capture specific binding partner or (2) detection agent or detection specific binding partner. In general, the linking agent can be any binding partner (preferably specific) that is immobilized on (attached to) the solid phase and that has the ability to immobilize the (1) capture agent or capture specific binding partner, or (2) detection agent or detection specific binding partner through a binding reaction. The linking agent enables the indirect binding of the capture agent to a solid phase material before the performance of the assay or during the performance of the assay. For examples, the solid phase can be plastic, derivatized plastic, magnetic, or non-magnetic metal, glass or silicon, including, for example, a test tube, microtiter well, sheet, bead, microparticle, chip, and other configurations known to those of ordinary skill in the art.

“Statistically significant” as used herein refers to the likelihood that a relationship between two or more variables is caused by something other than random chance. Statistical hypothesis testing is used to determine whether the result of a data set is statistically significant. In statistical hypothesis testing, a statistical significant result is attained whenever the observed p-value of a test statistic is less than the significance level defined of the study. The p-value is the probability of obtaining results at least as extreme as those observed, given that the null hypothesis is true. Examples of statistical hypothesis analysis include Wilcoxon signed-rank test, t-test, Chi-Square or Fisher's exact test. “Significant” as used herein refers to a change that has not been determined to be statistically significant (e.g., it may not have been subject to statistical hypothesis testing).

As used herein, “treating” prostate cancer refers to taking steps to obtain beneficial or desired results, including clinical results. Beneficial or desired clinical results include, but are not limited to, alleviation or amelioration of one or more symptoms associated with diseases or conditions.

As used herein, “administering” or “administration of” a compound or an agent to a subject can be carried out using one of a variety of methods known to those skilled in the art. For example, a compound or an agent can be administered, intravenously, arterially, intradermally, intramuscularly, intraperitoneally, intravenously, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow, or controlled release of the compound or agent. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. In some embodiments, a compound or an agent is administered orally, e.g., to a subject by ingestion, or intravenously, e.g., to a subject by injection. In some embodiments, the orally administered compound or agent is in an extended release or slow release formulation or administered using a device for such slow or extended release.

### DETAILED DESCRIPTION

The present invention relates to compositions and methods for assessing prostate cancer (e.g., identification of the aggressiveness or indolence of prostate cancer) in a subject. The compositions and methods include obtaining subject specific information (e.g., age, digital rectal exam (DRE) data, prostate volume, total prostate-specific antigen (PSA)) and obtaining a biological sample from a subject and determining a measurement for a panel of biomarkers in the biological sample.

The invention provides methods for identifying, assessing and/or predicting the aggressiveness or indolence of cancer (e.g., prostate cancer) in a subject (e.g., a subject suspected of having cancer, a subject diagnosed with a cancer, or a subject at risk for cancer). In some embodiments, the invention provides a method for identifying, assessing and/or predicting the aggressiveness or indolence of prostate cancer (e.g., in a patient previously diagnosed with prostate cancer).

Genomic expressions present in all cells within an individual are affected by and change consequent to a variety of factors. These factor include, but are not limited to, intrinsic inter-individual (e.g., gender, ethnic background, etc.) variations; age-related (temporal) variations; extracellular “milieu” stimuli (e.g., recent food/drink intake, recent vaccination, exposure to infectious organisms, etc.); the presence of one or more specific diseases (e.g., cancer, that a blood test aims to detect via detection of an immunological response); and other disease/conditions unrelated to the disease that conventional tests aim to detect. Each of these factors lead to an orchestrated upregulation and downregulation and silencing of certain genes.

Accordingly, in conventional blood-based disease assays (e.g., cancer assays, rheumatoid arthritis, infectious disease), a diseased patient's profile (e.g., from plasma, PBMCs, a WBC subpopulation, etc.) is compared to that of an individual identified not to have the disease (a control subject or panel of subjects) with the hope/expectation of identifying a disease signature. However, since the baseline/background signatures of the individual with the “Disease” are specific to his/her genomic profile and that of the “Control” are specific to his/her genomic profile, such intrinsic inter-individual differences have, and will always, impede the identification of a valid disease signature.

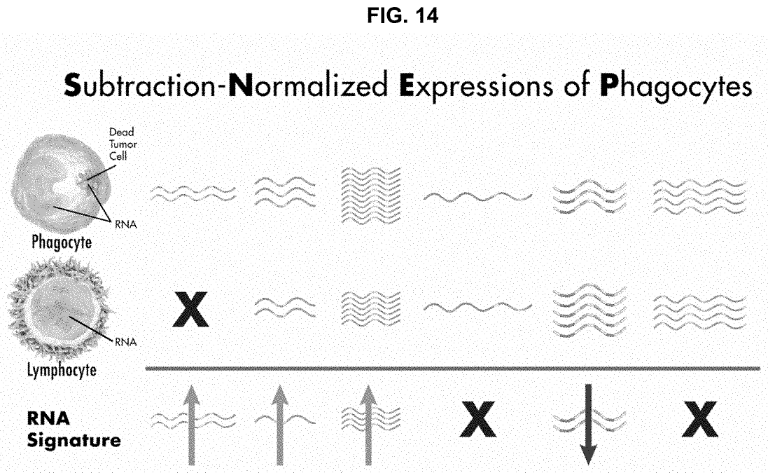

The invention provides assays utilizing Subtraction Normalized Expressions of Phagocytes (SNEP) to identify biomarkers (e.g., a nucleic acid, DNA, RNA, peptide, protein, or metabolite) that alone, or in combination with patient clinical information, find utility in the identification of a disease signature (e.g., that is used for detecting and/or identifying disease in a subject). In SNEP, intrinsic signatures not related to the disease are filtered out and the “normalized data” from the patient and the control are used to identify a disease specific signature. Thus, in some embodiments, the invention provides one or more disease signatures (e.g., one or more prostate cancer disease signatures) and methods of using the signature(s) to identify, assess, and/or predict various facets of disease in a subject. For example, in some embodiments, detecting or identifying disease in a subject comprises identifying, assessing and/or predicting the aggressiveness or indolence of cancer (e.g., prostate cancer) in a subject (e.g., in a patient previously diagnosed with prostate cancer) using one or more of the signatures described herein. The SNEP assay is schematically diagrammed in FIG. 14.

As described in the Examples, over one thousand patients were used to identify signatures of disease. CD2⁺ T cells and CD14⁺ monocytes and/or macrophages were isolated from patients, RNA extracted and whole genome, RNA sequencing performed (about 25,000 genes sequenced). The sequencing data was analyzed using an algorithm (sparse rank regression model with inputs including the weighted sum of clinical and sequencing transcript covariates) to generate receiver operating characteristic curves. Analysis generated during development of embodiments of the invention generated an Aggressiveness Index that aggregated maximum Gleason grade, number of positive biopsied cores, and maximum involvement among the biopsied cores (e.g., that provided the ability to discriminate, using one or more signatures identified herein, between aggressiveness or indolence of cancer (e.g., prostate cancer) in a subject).

Thus, an example of an Aggressiveness Index according to the invention utilized clinical parameters based on: maximum Gleason grade, maximal cross section surface area of a core, and number of positive cores to generate an aggressiveness index scored between 0-4, where a Score of 0 meant no evidence of cancer on 12 core or more biopsy; a Score of 1 meant low grade⁺ and low volume⁺ (i.e., Grade 1, 1-2 cores up to 10%; or Grade 2, 1-2 cores up to 5%); a Score of 2 meant low grade and low volume (i.e., Grade 1, 3-5 cores [20-40%]; or Grade 2, 3-4 cores [10-20%]; or Grade 3, 1-2 cores [1-5%]); a Score of 3 meant intermediate grade and intermediate volume (i.e., Grade 1, 6-12 cores [50-100%]; or Grade 2, 5-9 cores [30-70%]; or Grade 3, 3-6 cores [10-50%]; or Grade 4, 1-2 cores [1-5%]; or Grade 5, 1 core [1-2%]); and a Score of 4 meant high grade and high volume (i.e., Grade 2-3, >5 cores [>50%]; or Grade 4, >2 cores [>10%]; or Grade 5, >1 core [>1%]).

Throughout experiments conducted during development of embodiments of the invention (e.g., described in the Examples), a subset of inputs (biomarker and clinical covariates) were identified by the model as predictive, termed “PC signature”, that were solely responsible for the predictions made by the model, inputs not in the signature (with zero model coefficients), were ignored. After training the model on 713 patients, 61 covariates were identified having non-zero weights (See Table 1).

PC signatures identified contained multiple inputs, for example, clinical covariates including age, DRE, prostate volume, and total PSA, as well as biomarker covariates (See Table 1, Table 2, and Table 3).

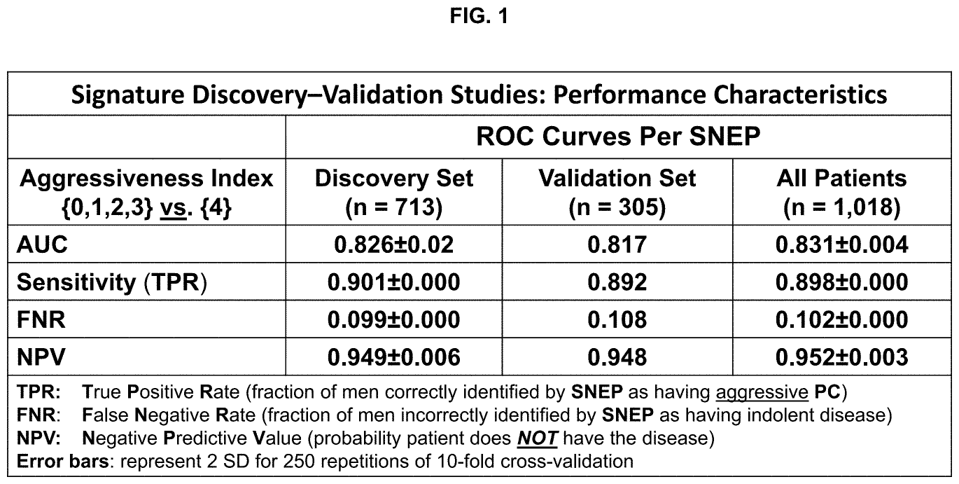

The performance characteristics of the model, in terms of Area Under the Receiving Operating Characteristic (AUROC), were evaluated on the remaining N=305 (30%) subjects, not used for model estimation, in order to obtain unbiased estimates of model performance (See FIG. 1, Validation Set). Multiple PC signatures were generated using this approach. For example, given the dataset of N=713 subjects, more than one combination of inputs (PC signatures) that yield comparatively similar performance metrics (statistically indifferent given the sample size) were made possible. Further, other inputs that correlated substantially with any of the elements of the signature can also be added to a modified, larger, signature without significantly impacting the performance characteristics of the model with the larger signature relative to the original.

Thus, in some embodiments, the invention utilizes SNEP assays and/or one or more signatures identified herein to stratify cancer patients. For example, in one embodiment, the invention provides SNEP assays and/or one or more signatures identified herein to stratify patients with indolent prostate disease from those with aggressive prostate cancer (e.g., that require life-saving treatments). Accordingly, in some embodiments, compositions and methods described herein find use in clinical assessment and management of subjects (e.g., patients at risk for cancer (e.g., prostate cancer)). For example, in some embodiments, SNEP assays and/or one or more signatures identified herein classify a patient as definitive for treatment (e.g., with one or more anti-cancer therapies) or as needing only surveillance (e.g., no treatment).

In some embodiments, compositions and methods of the invention (e.g., SNEP assays and/or one or more signatures identified herein) provide a clinician the ability to stratify a patient into either a treatment group (e.g., requiring cancer treatment and/or therapies) or a surveillance group (e.g., not requiring immediate treatment) without need for a physically invasive biopsy. That is, in some embodiments, compositions and methods of the invention are used to avoid unnecessary patient biopsies (e.g., prostate cancer biopsy), repeat biopsies, and/or the pain and suffering and risk factors/side effects consequent to biopsies (e.g., in men under active surveillance for prostate cancer). In some embodiments, compositions and methods of the invention benefit men diagnosed with prostate cancer in that the compositions and methods (SNEP assays and/or one or more signatures identified herein) identify patients needing further workup and/or treatment.

In one embodiment, the present invention provides biological markers and methods of using them to detect a cancer (e.g., prostate cancer). The present invention is based on the discovery that one or more markers selected from Table 1, Table 2, and/or Table 3 are useful in diagnosing prostate cancer, either alone, or when assessed in the context of one or more clinical covariates (e.g., age, digital rectal exam (DRE) data, prostate volume, total prostate-specific antigen (PSA)). In one embodiment, the invention provides a cancer (e.g., prostate cancer) aggressiveness index value (e.g., 0, 1, 2, 3, or 4) that identifies and characterizes cancer in a subject (e.g., scaled such that a value of 0 characterizes the absence of cancer in the subject ranging to a value of 4 that characterizes the presence of highly aggressive cancer in the subject). In some embodiments, one or more clinical covariates are concatenated with one or more biomarker levels and input into a sparse rank regression model in order to generate a prostate cancer aggressiveness index.

For example, in some embodiments, by measuring the levels of the biomarkers (e.g., gene expression levels, protein expression levels, or protein activity levels) in a population of phagocytes (e.g., macrophages, monocytes, or neutrophils) from a human subject, one can provide a reliable diagnosis for prostate cancer (e.g., identifying, assessing and/or predicting the aggressiveness or indolence of prostate cancer).

As used herein, a “level” of a marker of this invention can be qualitative (e.g., presence or absence) or quantitative (e.g., amounts, copy numbers, or dosages). In some embodiments, a level of a marker at a zero value can indicate the absence of this marker. The levels of any marker of this invention can be measured in various forms. For example, the level can be a gene expression level, a RNA transcript level, a protein expression level, a protein activity level, an enzymatic activity level.

The markers of this invention can be used in methods for diagnosing or aiding in the diagnosis of prostate cancer by comparing levels (e.g., gene expression levels, or protein expression levels, or protein activities) of one or more prostate cancer markers (e.g., nucleic acids or proteins) between phagocytes (e.g., macrophages, monocytes, or neutrophils) and non-phagocytic cells (e.g., T cells) taken from the same individual. This invention also provides methods for assessing the risk of developing prostate cancer, prognosing the cancer, monitoring the cancer progression or regression, assessing the efficacy of a treatment, or identifying a compound capable of ameliorating or treating the cancer.

Compositions and methods of the invention find use in the identification, characterization, and classification (e.g., via computing aggressiveness index) of cancer in a subject. In particular embodiments, the compositions and methods of the invention are applied to prostate cancer. As used herein, “prostate cancer” means any cancer of the prostate including, but not limited to, adenocarcinoma and small cell carcinoma.

In some embodiments, the invention provides a method for identifying, assessing and/or predicting the aggressiveness or indolence of cancer (e.g., prostate cancer) in a subject (e.g., a subject suspected of having cancer, a subject diagnosed with a cancer, or a subject at risk for cancer). In some embodiments, the invention provides a method for identifying, assessing and/or predicting the aggressiveness or indolence of prostate cancer (e.g., in a patient previously diagnosed with prostate cancer).

In some embodiments, the invention provides a method of measuring a panel of biomarkers in a subject comprising obtaining a biological sample from the subject; determining a measurement for the panel of biomarkers in the biological sample, wherein the panel of biomarkers comprise one or more (e.g., two, three, four, five, six, seven, eight, nine, ten, eleven, twelve, thirteen, fourteen, fifteen or more) biomarkers of Table 1, Table 2, and/or Table 3 and wherein the measurement comprises measuring a level of each of the biomarkers in the panel. In some embodiments, measuring the panel of biomarkers in the subject identifies, assesses, and/or predicts the aggressiveness or indolence of cancer (e.g., prostate cancer) in a subject (e.g., a subject suspected of having cancer, a subject diagnosed with a cancer, or a subject at risk for cancer). In some embodiments, the biological sample comprises CD2⁺ cells and/or CD14⁺ cells. In one embodiment, determining a measurement for the panel of biomarkers in the biological sample comprises measuring a level of each of the biomarkers in the panel in CD2⁺ cells and/or CD14⁺ cells. In one embodiment, the method further comprises obtaining one or more clinical data from the subject selected from the group consisting of age, race, digital rectal exam (DRE), prostate volume, and total prostate-specific antigen (PSA). In some embodiments, the one or more clinical data are used as clinical covariates and concatenated with the biomarker levels and input into a sparse rank regression model (e.g., in order to identify, assess, and/or predict the aggressiveness or indolence of cancer (e.g., prostate cancer) in a subject). In some embodiments, measuring a level of each of the biomarkers in the panel comprises measuring gene expression levels.

In some embodiments, the invention provides a method of measuring a panel of biomarkers in a subject comprising obtaining a biological sample from the subject; determining a measurement for the panel of biomarkers in the biological sample, wherein the panel of biomarkers comprise one or more (e.g., two, three, four, five, six, seven, eight, nine, ten, eleven, twelve, thirteen, fourteen, or fifteen) biomarkers selected from of C11orf94, C9orf135, DSP, EGFL6, FST, FSTL1, GATA2, GRID1, KLF17, KRTAP5-8, MID1, MYO1D, OOEP, RSPH9 and TAGLN3, and wherein the measurement comprises measuring a level of each of the biomarkers in the panel. In some embodiments, measuring the panel of biomarkers in the subject identifies, assesses, and/or predicts the aggressiveness or indolence of cancer (e.g., prostate cancer) in a subject (e.g., a subject suspected of having cancer, a subject diagnosed with a cancer, or a subject at risk for cancer). In some embodiments, the biological sample comprises CD2⁺ cells and/or CD14⁺ cells. In some embodiments, determining a measurement for the panel of biomarkers in the biological sample comprises measuring a level of each of the biomarkers in the panel in CD2⁺ cells and/or CD14⁺ cells. In one embodiment, the method further comprises obtaining one or more clinical data from the subject selected from the group consisting of age, race, digital rectal exam (DRE), prostate volume, and total prostate-specific antigen (PSA). In some embodiments, the one or more clinical data are used as clinical covariates and concatenated with the biomarker levels and input into a sparse rank regression model (e.g., in order to identify, assess, and/or predict the aggressiveness or indolence of cancer (e.g., prostate cancer) in a subject). In some embodiments, measuring a level of each of the biomarkers in the panel comprises measuring gene expression levels.

The invention is not limited by how gene expression levels are measured. Indeed, any means of measuring gene expression levels may be used including, but not limited to, polymerase chain reaction (PCR) analysis, sequencing analysis, electrophoretic analysis, restriction fragment length polymorphism (RFLP) analysis, Northern blot analysis, quantitative PCR, reverse-transcriptase-PCR analysis (RT-PCR), allele-specific oligonucleotide hybridization analysis, comparative genomic hybridization, heteroduplex mobility assay (HMA), single strand conformational polymorphism (SSCP), denaturing gradient gel electrophoresis (DGGE), RNAase mismatch analysis, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), surface plasmon resonance, Southern blot analysis, in situ hybridization, fluorescence in situ hybridization (FISH), chromogenic in situ hybridization (CISH), immunohistochemistry (IHC), microarray, comparative genomic hybridization, karyotyping, multiplex ligation-dependent probe amplification (MLPA), Quantitative Multiplex PCR of Short Fluorescent Fragments (QMPSF), microscopy, methylation specific PCR (MSP) assay, HpaII tiny fragment Enrichment by Ligation-mediated PCR (HELP) assay, radioactive acetate labeling assays, colorimetric DNA acetylation assay, chromatin immunoprecipitation combined with microarray (ChIP-on-chip) assay, restriction landmark genomic scanning, Methylated DNA immunoprecipitation (MeDIP), molecular break light assay for DNA adenine methyltransferase activity, chromatographic separation, methylation-sensitive restriction enzyme analysis, bisulfite-driven conversion of non-methylated cytosine to uracil, methyl-binding PCR analysis, or a combination thereof. In some embodiments, gene expression levels are measured by a sequencing technique such as, but not limited to, direct sequencing, RNA sequencing, whole transcriptome shotgun sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, mass spectrometry, and a combination thereof. In some embodiments, measuring a level of each of the biomarkers in the panel comprises measuring protein expression levels.

The invention is not limited to any particular method of measuring protein expression levels. Exemplary methods of measuring protein expression levels include, but are not limited to, an immunohistochemistry assay, an enzyme-linked immunosorbent assay (ELISA), in situ hybridization, chromatography, liquid chromatography, size exclusion chromatography, high performance liquid chromatography (HPLC), gas chromatography, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), radioimmunoassays, microscopy, microfluidic chip-based assays, surface plasmon resonance, sequencing, Western blotting assay, or a combination thereof. In some embodiments, measuring a level of each of the biomarkers in the panel comprises measuring by a qualitative assay, a quantitative assay, or a combination thereof. Exemplary quantitative assays include, but are not limited to, sequencing, direct sequencing, RNA sequencing, whole transcriptome shotgun sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), polymerase chain reaction (PCR) analysis, quantitative PCR, real-time PCR, fluorescence assay, colorimetric assay, chemiluminescent assay, or a combination thereof. In some embodiments, the subject is a human.

The invention also provides a kit for performing measurement at least two (e.g., two, three, four, five, six, seven, eight, nine, ten, eleven, twelve or more) of the markers listed in Table 1, Table 2, and/or Table 3, wherein the kit comprises reagents for measuring the at least two markers.

In another aspect, the methods (e.g., diagnosis of prostate cancer, prognosis of prostate cancer, or assessing the risk of developing prostate cancer) provided in the invention comprise: a) measuring the levels of one or more (e.g., two, three, four, five, six, seven, eight, nine, ten, eleven, twelve, thirteen, fourteen, fifteen or more) markers selected from Table 1, Table 2, and/or Table 3 in a population of a subject's macrophage or monocyte cells; b) measuring the levels of one or more of the selected markers in a population of a subject's non-phagocytic cells (e.g., T-cells, B-cells, null cells, basophils or the mixtures of two more non-phagocytic cells); comparing the measured levels in step a) to the measured levels in step b) and further identifying a difference between the measured levels of a) and b). The identified difference is indicative of the diagnosis (e.g., presence or absence), prognosis (e.g., lethal outcome, or tumor stage), or the risk of developing prostate cancer.

In another aspect, the methods (e.g., diagnosis of prostate cancer, prognosis of prostate cancer, or assessing the risk of developing prostate cancer) provided in the invention comprise: a) measuring the levels of one or more (e.g., two, three, four, five, six, seven, eight, nine, ten, eleven, twelve, thirteen, fourteen, fifteen or more) markers selected from Table 1, Table 2, and/or Table 3 in a population of a subject's macrophage or monocyte cells; identifying a difference between the measured levels of the selected markers in step a) and the levels of the selected markers in a control (e.g., a healthy control cell, or a control cell from a healthy subject). The identified difference is indicative of the diagnosis (e.g., presence or absence), prognosis (e.g., lethal outcome, or tumor stage), or the risk of developing prostate cancer. In some embodiments, the selected markers are up-regulated in prostate cancer patients. In some embodiments, the selected markers are down-regulated in prostate cancer patients. In some embodiments, the selected markers comprise at least one marker that is up-regulated and at least one marker that is down-regulated. In some embodiments, the method of diagnosing, prognosing, and/or assessing the aggressiveness and/or indolence of prostate cancer provided by the invention (e.g., via measuring the levels of one or more markers selected from Table 1, Table 2, and/or Table 3 optionally in combination with one or more clinical covariates) provides a better diagnostic, prognostic and/or assessment than a Gleason score of the prostate cancer.

In another aspect, the invention provides methods for assessing the efficacy of a treatment for prostate cancer, monitoring the progression or regression of prostate cancer, or identifying a compound capable of ameliorating or treating prostate cancer, respectively, in a subject comprising: a) measuring the levels of one or more (e.g., two, three, four, five, six, seven, eight, nine, ten, eleven, twelve, thirteen, fourteen, fifteen or more) markers selected from Table 1, Table 2, and/or Table 3 in a population of the subject's macrophage or monocyte cells before the treatment, or at a first time point, or before administration of the compound, respectively; b) measuring the levels of the one or more selected markers in a population of the subject's non-phagocytic cells before the treatment, or at the first time point, or before administration of the compound, respectively; c) identifying a first difference between the measured levels of the one or more selected markers in steps a) and b); d) measuring the levels of the one or more selected markers in a population of the subject's macrophage or monocyte cells after the treatment, or at a second time point, or after administration of the compound, respectively; e) measuring the levels of the one or more selected markers in a population of the subject's non-phagocytic cells after the treatment, or at the second time point, or after administration of the compound, respectively; f) identifying a second difference between the measured levels of the one or more selected markers in steps d) and e); and g) identifying a difference between the first difference and the second difference, wherein the difference identified in g) is indicative of the efficacy of the treatment for the prostate cancer, or the progression or regression of the prostate cancer, or whether the compound is capable of ameliorating or treating the prostate cancer, respectively, in the subject.

In another aspect, the invention provides methods for assessing the efficacy of a treatment for prostate cancer, monitoring the progression or regression of prostate cancer, or identifying a compound capable of ameliorating or treating prostate cancer, respectively, in a subject comprising: a) measuring the levels of one or more (e.g., two, three, four, five, six, seven, eight, nine, ten, eleven, twelve, thirteen, fourteen, fifteen or more) markers selected from Table 1, Table 2, and/or Table 3 in a population of the subject's macrophage or monocyte cells before the treatment, or at a first time point, or before administration of the compound, respectively; b) identifying a first difference between the measured levels of the one or more selected markers in step (a) and the levels of the one or more selected markers in a control (e.g., a healthy control cell, or a control cell from a healthy subject) before the treatment, or at the first time point, or before administration of the compound, respectively; c) measuring the levels of the one or more selected markers in a population of the subject's macrophage or monocyte cells after the treatment, or at a second time point, or after administration of the compound, respectively; d) identifying a second difference between the measured levels of the one or more selected markers in step c) and the levels of the one or more selected markers in a control after the treatment, or at the second time point, or after administration of the compound, respectively; and e) identifying a difference between the first difference and the second difference, wherein the difference identified in e) is indicative of the efficacy of the treatment for the prostate cancer, or the progression or regression of the prostate cancer, or whether the compound is capable of ameliorating or treating the prostate cancer, respectively, in the subject.

In some embodiments, two sub-populations of phagocytic cells (e.g., monocytes) are used in the methods of this invention, i.e., phagocytic cells that have a DNA content greater than 2n (the >2n phagocytic cells) and phagocytic cells that have a DNA content of 2n (the =2n phagocytic cells). In such embodiments, the levels of the selected markers in the >2n phagocytic cells are compared to the =2n phagocytic cells to identify one or more differences. The identified differences indicate whether the subject has prostate cancer, or has a risk of developing prostate cancer, or has a progressing or progressive prostate cancer.

In some embodiments, the levels of two, three, four, five, six, seven, eight, nine, ten, eleven, twelve, thirteen, fourteen, fifteen, or more of the markers selected from Table 1 are measured. In some embodiments, the levels of one or more (e.g., two, three, four, five, six, seven, eight, nine, ten, eleven, twelve, thirteen, fourteen, fifteen or more) markers selected from Table 1, Table 2, and/or Table 3 are measured, and are concatenated with one or more clinical data (clinical covariates) and input into a sparse rank regression model/algorithm in order to identify, assess and/or predict the aggressiveness or indolence of cancer (e.g., prostate cancer) in a subject (e.g., a subject suspected of having cancer, a subject diagnosed with a cancer, or a subject at risk for cancer). The invention is not limited by the type of clinical data utilized. Indeed, a variety of clinical data may be used including, but not limited to, age, race, digital rectal exam (DRE), prostate volume, total prostate-specific antigen (PSA), tumor stage, tumor grade, tumor size, tumor visual characteristics, tumor growth, tumor thickness, tumor progression, tumor metastasis, tumor distribution within the body, odor, molecular pathology, genomics, and/or tumor angiograms.

In some embodiments, at least one or more of the selected markers from Table 1, Table 2, and/or Table 3 may be substituted with a biological marker different from any of the selected markers. In some embodiments, such biological markers may be known markers for prostate cancer. In some embodiments, such biological markers and the substituted selected markers may belong to the same signaling or biological pathway (e.g., a protein synthesis pathway, Thl cytokine production pathway, transcription pathway, programmed cell death pathway), or may have similar biological function or activity (e.g., protein synthesis, Thl cytokine production, nucleotide binding, protein binding, transcription, a receptor for purines coupled to G-proteins, inhibition of programmed cell death, neutrophil activation, an IL-8 receptor, an HSP70-interacting protein, stimulating ATPase activity), or may be regulated by a common protein, or may belong to the same protein complex (e.g., an HSP70 protein complex). In some embodiments, at least one or more of the selected markers from Table 2 or Table 3 is substituted with a biological marker from Table 1.

In some embodiments, a population of a subject's macrophage, monocyte, and/or neutrophil cells is used as the selected phagocytic cells for measuring the levels of the selected markers and a population of the subject's T-cells is used as the selected non-phagocytic cells for measuring the levels of the selected markers. In other embodiments, a population of the subject's neutrophil cells is used as the selected phagocytic cells for measuring the levels of the selected markers and a population of the subject's T-cells is used as the selected non-phagocytic cells for measuring the levels of the selected markers.

The gene names/descriptions provided in Table 1, Table 2, and/or Table 3 are merely illustrative. The markers of this invention encompass all forms and variants of any specifically described markers, including, but not limited to, polymorphic or allelic variants, isoforms, mutants, derivatives, precursors including nucleic acids and pro-proteins, cleavage products, and structures comprised of any of the markers as constituent subunits of the fully assembled structure.

Each embodiment described herein may be combined with any other embodiment described herein.

Methods using the prostate cancer markers described herein provide high specificity, sensitivity, and accuracy in detecting and diagnosing prostate cancer. The methods also eliminate the “inequality of baseline” that is known to occur among individuals due to intrinsic (e.g., age, gender, ethnic background, health status and the like) and temporal variations in marker expression. Additionally, by using a comparison of phagocytes and non-phagocytes from the same individual, the methods also allow detection, diagnosis, and treatment to be personalized to the individual. Accordingly, in some embodiments, the invention provides non-invasive assays for the early detection of prostate cancer, i.e., before the prostate cancer can be diagnosed by conventional diagnostic techniques, e.g., imaging techniques, and, therefore, provide a foundation for improved decision-making relative to the needs and strategies for intervention, prevention, and treatment of individuals with such disease or condition.

The methods described herein are supported by whole genome microarray data of total RNA samples isolated from phagocytic cells (e.g., macrophages, monocytes, dendritic cells, and/or neutrophils) and from non-phagocytic cells (e.g., T cells). The samples were obtained from human subjects with and without prostate cancer. The data from these microarray experiments demonstrate that macrophage/monocyte-T cell comparisons easily and accurately differentiate between prostate cancer patients and human subjects without prostate cancer.

The methods of this invention can be used together with any known diagnostic methods, such as physical inspection, visual inspection, biopsy, scanning, histology, radiology, imaging, ultrasound, use of a commercial kit, genetic testing, immunological testing, analysis of bodily fluids, or monitoring neural activity.

Phagocytic cells that can be used in the methods of this invention include all types of cells that are capable of ingesting various types of substances (e.g., apoptotic cells, infectious agents, dead cells, viable cells, cell-free DNAs, cell-free RNAs, cell-free proteins). In some embodiments, the phagocytic cells are neutrophils, macrophages, monocytes, dendritic cells, foam cells, mast cells, eosinophils, or keratinocytes. In some embodiments, the phagocytic cells can be a mixture of different types of phagocytic cells. In some embodiments, the phagocytic cells can be activated phagocytic cells, e.g., activated macrophages, monocytes, or neutrophils. In some embodiments, a phagocyte is a histiocyte, e.g., a Langerhans cell.

In certain embodiments, markers used in the methods of invention are up-regulated or activated in phagocytes (e.g., macrophages, monocytes, or neutrophils) compared to non-phagocytes. In certain embodiments, markers used in the methods of invention are down-regulated or inhibited in phagocytes (e.g., macrophages, monocytes, or neutrophils) compared to non-phagocytes. As used herein, “up-regulation or up-regulated” can refer to an increase in expression levels (e.g., gene expression or protein expression), gene copy numbers, gene dosages, and other qualitative or quantitative detectable state of the markers. Similarly, “down-regulation or down-regulated” can refer to a decrease in expression levels, gene copy numbers, gene dosages, and other qualitative or quantitative detectable state of the markers. As used herein, “activation or activated” can refer to an active state of the marker, e.g., a phosphorylation state, a DNA methylation state, or a DNA acetylation state. Similarly, “inhibition or inhibited” can refer to a repressed state or an inactivated state of the marker, e.g., a de-phosphorylation state, a ubiquitination state, or a DNA de-methylation state.

In certain embodiments, methods of this invention also comprise at least one of the following steps before determination of various levels: i) lysing the phagocytic or non-phagocytic cells; and ii) extracting cellular contents from the lysed cells. Any known cell lysis and extraction methods can be used herein. In certain embodiments, at least one or more prostate cancer markers are present in the phagocytes. In certain embodiments, there is no marker present in the cellular contents of the non-phagocytic cells.

In certain embodiments, the phagocytic cells and/or non-phagocytic cells are isolated from a bodily fluid sample, tissues, or population of cells. Exemplary bodily fluid samples can be whole blood, urine, stool, saliva, lymph fluid, cerebrospinal fluid, synovial fluid, cystic fluid, ascites, pleural effusion, fluid obtained from a pregnant woman in the first trimester, fluid obtained from a pregnant woman in the second trimester, fluid obtained from a pregnant woman in the third trimester, maternal blood, amniotic fluid, chorionic villus sample, fluid from a preimplantation embryo, maternal urine, maternal saliva, placental sample, fetal blood, lavage and cervical vaginal fluid, interstitial fluid, buccal swab sample, sputum, bronchial lavage, Pap smear sample, or ocular fluid. In some embodiments, the phagocytic cells or non-phagocytic cells are isolated from white blood cells.

In the methods of this invention, cell separation/isolation/purification methods are used to isolate populations of cells from bodily fluid sample, cells, or tissues of a subject. A skilled worker can use any known cell separation/isolation/purification techniques to isolate phagocytic cells and non-phagocytic cells from a bodily fluid. Exemplary techniques include, but are not limited to, using antibodies, flow cytometry, fluorescence activated cell sorting, filtration, gradient-based centrifugation, elution, microfluidics, immunomagnetic separation technique, multiple size immuno-beads filtration techniques, fluorescent-magnetic separation technique, nanostructure, quantum dots, high throughput microscope-based platform, or a combination thereof.

In certain embodiments, the phagocytic cells and/or non-phagocytic cells are isolated by using a product secreted by the cells. In certain embodiments, the phagocytic cells and/or non-phagocytic cells are isolated by using a cell surface target (e.g., receptor protein) on the surface of the cells. In some embodiments, the cell surface target is a protein that has been engulfed by phagocytic cells. In some embodiments, the cell surface target is expressed by cells on their plasma membranes. In some embodiments, the cell surface target is an exogenous protein that is translocated on the plasma membranes, but not expressed by the cells (e.g., the phagocytic cells). In some embodiments, the cell surface target is a marker of prostate cancer.

In certain aspects of the methods described herein, analytes include nucleic acids, proteins, or any combinations thereof. In certain aspects of the methods described herein, markers include nucleic acids, proteins, or any combinations thereof. As used herein, the term “nucleic acid” is intended to include DNA molecules (e.g., cDNA or genomic DNA), RNA molecules (e.g., mRNA), DNA-RNA hybrids, and analogs of the DNA or RNA generated using nucleotide analogs. The nucleic acid molecule can be a nucleotide, oligonucleotide, double-stranded DNA, single-stranded DNA, multi-stranded DNA, complementary DNA, genomic DNA, non-coding DNA, messenger RNA (mRNAs), microRNA (miRNAs), small nucleolar RNA (snoRNAs), ribosomal RNA (rRNA), transfer RNA (tRNA), small interfering RNA (siRNA), heterogeneous nuclear RNAs (hnRNA), or small hairpin RNA (shRNA). In some embodiments, the nucleic acid is a transrenal nucleic acid. A transrenal nucleic acid is an extracellular nucleic acid that is excreted in the urine. See, e.g., U.S. Patent Publication No. 20100068711 and U.S. Patent Publication No. 20120021404.

As used herein, the term “amino acid” includes organic compounds containing both a basic amino group and an acidic carboxyl group. Included within this term are natural amino acids (e.g., L-amino acids), modified and unusual amino acids (e.g., D-amino acids and .beta.-amino acids), as well as amino acids which are known to occur biologically in free or combined form but usually do not occur in proteins. Natural protein occurring amino acids include alanine, arginine, asparagine, aspartic acid, cysteine, glutamic acid, glutamine, glycine, histidine, isoleucine, leucine, lysine, methionine, phenylalanine, serine, threonine, tyrosine, tryptophan, proline, and valine. Natural non-protein amino acids include arginosuccinic acid, citrulline, cysteine sulfuric acid, 3,4-dihydroxyphenylalanine, homocysteine, homoserine, ornithine, 3-monoiodotyrosine, 3,5-diiodotryosine, 3,5,5-triiodothyronine, and 3,3′,5,5′-tetraiodothyronine. Modified or unusual amino acids include D-amino acids, hydroxylysine, 4-hydroxyproline, N-Cbz-protected amino acids, 2,4-diaminobutyric acid, homoarginine, norleucine, N-methylaminobutyric acid, naphthylalanine, phenylglycine, .alpha.-phenylproline, tert-leucine, 4-aminocyclohexylalanine, N-methyl-norleucine, 3,4-dehydroproline, N,N-dimethylaminoglycine, N-methylaminoglycine, 4-aminopiperidine-4-carboxylic acid, 6-aminocaproic acid, trans-4-(aminomethyl)-cyclohexanecarboxylic acid, 2-, 3-, and 4-(aminomethyl)-benzoic acid, 1-aminocyclopentanecarboxylic acid, 1-aminocyclopropanecarboxylic acid, and 2-benzyl-5-aminopentanoic acid.

As used herein, the term “peptide” includes compounds that consist of two or more amino acids that are linked by means of a peptide bond. Peptides may have a molecular weight of less than 10,000 Daltons, less than 5,000 Daltons, or less than 2,500 Daltons. The term “peptide” also includes compounds containing both peptide and non-peptide components, such as pseudopeptide or peptidomimetic residues or other non-amino acid components. Such compounds containing both peptide and non-peptide components may also be referred to as a “peptide analog.”

As used herein, the term “protein” includes compounds that consist of amino acids arranged in a linear chain and joined together by peptide bonds between the carboxyl and amino groups of adjacent amino acid residues. Proteins used in methods of the invention include, but are not limited to, amino acids, peptides, antibodies, antibody fragments, cytokines, lipoproteins, or glycoproteins.

As used herein, the term “antibody” includes polyclonal antibodies, monoclonal antibodies (including full length antibodies which have an immunoglobulin Fc region), antibody compositions with polyepitopic specificity, multispecific antibodies (e.g., bispecific antibodies, diabodies, and single-chain molecules, and antibody fragments (e.g., Fab or F(ab′).sub.2, and Fv). For the structure and properties of the different classes of antibodies, see e.g., Basic and Clinical Immunology, 8th Edition, Daniel P. Sties, Abba I. Ten and Tristram G. Parsolw (eds), Appleton & Lange, Norwalk, Conn., 1994, page 71 and Chapter 6.

As used herein, the term “cytokine” refers to a secreted protein or active fragment or mutant thereof that modulates the activity of cells of the immune system. Examples of cytokines include, without limitation, interleukins, interferons, chemokines, tumor necrosis factors, colony-stimulating factors for immune cell precursors, and the like.

As used herein, the term “lipoprotein” includes negatively charged compositions that comprise a core of hydrophobic cholesteryl esters and triglyceride surrounded by a surface layer of amphipathic phospholipids with which free cholesterol and apolipoproteins are associated. Lipoproteins may be characterized by their density (e.g. very-low-density lipoprotein (VLDL), low-density lipoprotein (LDL) and high density lipoprotein (HDL)), which is determined by their size, the relative amounts of lipid and protein. Lipoproteins may also be characterized by the presence or absence of particular modifications (e.g. oxidization, acetylation, or glycation).

As used herein, the term “glycoprotein” includes glycosides which have one or more oligo- or polysaccharides covalently attached to a peptide or protein. Exemplary glycoproteins can include, without limitation, immunoglobulins, members of the major histocompatibility complex, collagens, mucins, glycoprotein IIb/IIIa, glycoprotein-41 (gp41) and glycoprotein-120 (gp12), follicle-stimulating hormone, alpha-fetoprotein, erythropoietin, transferrins, alkaline phosphatase, and lectins.

In some embodiments of the invention, a sample may comprise one or more stabilizers for a cell or an analyte such as DNA, RNA, and/or protein. For example, a sample may comprise a DNA stabilizer, an RNA stabilizer, and/or a protein stabilizer. Stabilizers are well known in the art and include, for example, DNAse inhibitors, RNAse inhibitors, and protease inhibitors or equivalents thereof.

In some embodiments of the invention, levels of at least one or more prostate cancer markers are compared. This comparison can be quantitative or qualitative. Quantitative measurements can be taken using any of the assays described herein. For example, sequencing, direct sequencing, random shotgun sequencing, Sanger dideoxy termination sequencing, targeted sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), polymerase chain reaction (PCR) analysis, quantitative PCR, real-time PCR, fluorescence assay, colorimetric assay, chemiluminescent assay, or a combination thereof.

Quantitative comparisons can include statistical analyses such as t-test, ANOVA, Krustal-Wallis, Wilcoxon, Mann-Whitney, and odds ratio. Quantitative differences can include differences in the levels of markers between levels or differences in the numbers of markers present between levels, and combinations thereof. Examples of levels of the markers can be, without limitation, gene expression levels, nucleic acid levels, and protein levels. Qualitative differences can include, but are not limited to, activation and inactivation, protein degradation, nucleic acid degradation, and covalent modifications.

In certain embodiments of the invention, the level is a nucleic acid level or a protein level, or a combination thereof. The level can be qualitatively or quantitatively determined.

A nucleic acid level can be, without limitation, a genotypic level, a single nucleotide polymorphism level, a gene mutation level, a gene copy number level, a DNA methylation level, a DNA acetylation level, a chromosome dosage level, a gene expression level, or a combination thereof.

The nucleic acid level can be determined by any methods known in the art to detect genotypes, single nucleotide polymorphisms, gene mutations, gene copy numbers, DNA methylation states, DNA acetylation states, chromosome dosages. Exemplary methods include, but are not limited to, polymerase chain reaction (PCR) analysis, sequencing analysis, electrophoretic analysis, restriction fragment length polymorphism (RFLP) analysis, Northern blot analysis, quantitative PCR, reverse-transcriptase-PCR analysis (RT-PCR), allele-specific oligonucleotide hybridization analysis, comparative genomic hybridization, heteroduplex mobility assay (HMA), single strand conformational polymorphism (SSCP), denaturing gradient gel electrophoresis (DGGE), RNAase mismatch analysis, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), surface plasmon resonance, Southern blot analysis, in situ hybridization, fluorescence in situ hybridization (FISH), chromogenic in situ hybridization (CISH), immunohistochemistry (IHC), microarray, comparative genomic hybridization, karyotyping, multiplex ligation-dependent probe amplification (MLPA), Quantitative Multiplex PCR of Short Fluorescent Fragments (QMPSF), microscopy, methylation specific PCR (MSP) assay, HpaII tiny fragment Enrichment by Ligation-mediated PCR (HELP) assay, radioactive acetate labeling assays, colorimetric DNA acetylation assay, chromatin immunoprecipitation combined with microarray (ChIP-on-chip) assay, restriction landmark genomic scanning, Methylated DNA immunoprecipitation (MeDIP), molecular break light assay for DNA adenine methyltransferase activity, chromatographic separation, methylation-sensitive restriction enzyme analysis, bisulfite-driven conversion of non-methylated cytosine to uracil, co-amplification at lower denaturation temperature-PCR (COLD-PCR), multiplex PCR, methyl-binding PCR analysis, or a combination thereof.

As used herein, the term “sequencing” is used in a broad sense and refers to any technique known in the art that allows the order of at least some consecutive nucleotides in at least part of a nucleic acid to be identified, including without limitation at least part of an extension product or a vector insert. Exemplary sequencing techniques include targeted sequencing, single molecule real-time sequencing, whole transcriptome shotgun sequencing (“RNA-seq”), electron microscopy-based sequencing, transistor-mediated sequencing, direct sequencing, random shotgun sequencing, Sanger dideoxy termination sequencing, exon sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, mass spectrometry, and a combination thereof. In some embodiments, sequencing comprises an detecting the sequencing product using an instrument, for example but not limited to an ABI PRISM™ 377 DNA Sequencer, an ABI PRISM′ 310, 3100, 3100-Avant, 3730, or 3730xI Genetic Analyzer, an ABI PRISM′ 3700 DNA Analyzer, or an Applied Biosystems SOLiD™ System (all from Applied Biosystems), a Genome Sequencer 20 System (Roche Applied Science), or a mass spectrometer. In certain embodiments, sequencing comprises emulsion PCR. In certain embodiments, sequencing comprises a high throughput sequencing technique, for example but not limited to, massively parallel signature sequencing (MPSS).

In further embodiments of the invention, a protein level can be a protein expression level, a protein activation level, or a combination thereof. In some embodiments, a protein activation level can comprise determining a phosphorylation state, an ubiquitination state, a myristylation state, or a conformational state of the protein.

A protein level can be detected by any methods known in the art for detecting protein expression levels, protein phosphorylation state, protein ubiquitination state, protein myristylation state, or protein conformational state. In some embodiments, a protein level can be determined by an immunohistochemistry assay, an enzyme-linked immunosorbent assay (ELISA), in situ hybridization, chromatography, liquid chromatography, size exclusion chromatography, high performance liquid chromatography (HPLC), gas chromatography, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), radioimmunoassays, microscopy, microfluidic chip-based assays, surface plasmon resonance, sequencing, Western blotting assay, or a combination thereof.

As used herein, the “difference” between different levels detected by the methods of this invention can refer to different gene copy numbers, different DNA, RNA, or protein expression levels, different DNA methylation states, different DNA acetylation states, and different protein modification states. The difference can be a difference greater than 1 fold (e.g., 1.0 to 100.0 fold, or greater). In some embodiments, the difference is a 1.05-fold, 1.1-fold, 1.2-fold, 1.3-fold, 1.4-fold, 1.5-fold, 2-fold, 2.5-fold, 3-fold, 4-fold, 5-fold, 6-fold, 7-fold, 8-fold, 9-fold, or 10-fold difference. In some embodiments, the difference is any fold difference between 1-10, 2-10, 5-10, 10-20, or 10-100 fold.

In some embodiments, the difference is differential gene expression (DGE), e.g. DGE of phagocytes vs. non-phagocytes. DGE can be measured as X=log 2(Y_{P})−log 2(Y_{NP}). The DGE may be any number, provided that it is significantly different between the phagocytes and the non-phagocytes. For example, a 2-fold increase in gene expression could be represented as X=log₂ (Y_{P})−log 2(Y_{NP})=log₂(Y_{P/Y2NP})=log₂(2)=1, while a 2-fold decrease in gene expression could be represented as X=log₂(Y_{P})−log₂(Y_{NP})=log₂ (Y_{2P/YNP})=log₂(1/2)=−1. Down-regulated genes have X<0, while up-regulated genes have X>0. See, e.g., Efron, J Am Stat Assoc 104:1015-1028 (2009).

A general principle of assays to detect markers involves preparing a sample or reaction mixture that may contain the marker (e.g., one or more of DNA, RNA, or protein) and a probe under appropriate conditions and for a time sufficient to allow the marker and probe to interact and bind, thus forming a complex that can be removed and/or detected in the reaction mixture. These assays can be conducted in a variety of ways.

For example, one method to conduct such an assay would involve anchoring the marker or probe onto a solid phase support, also referred to as a substrate, and detecting target marker/probe complexes anchored on the solid phase at the end of the reaction. In one embodiment of such a method, a sample from a subject, which is to be assayed for presence and/or concentration of marker, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, marker or probe molecules which are immobilized through conjugation of biotin and streptavidin. Such biotinylated assay components can be prepared from biotin-NHS(N-hydroxysuccinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the marker or probe belongs. Well known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of marker/probe complexes anchored to the solid phase can be accomplished in a number of methods outlined herein.

In certain exemplary embodiments, the probe, when it is the unanchored assay component, can be labeled for the purpose of detection and readout of the assay, either directly or indirectly, with detectable labels discussed herein and which are well-known to one skilled in the art.

It is also possible to directly detect marker/probe complex formation without further manipulation or labeling of either component (marker or probe), for example by utilizing the technique of fluorescence energy transfer (see, for example, U.S. Pat. Nos. 5,631,169 and 4,868,103). A fluorophore label on the first, donor molecule is selected such that, upon excitation with incident light of appropriate wavelength, its emitted fluorescent energy will be absorbed by a fluorescent label on a second acceptor molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a marker can be accomplished without labeling either assay component (probe or marker) by utilizing a technology such as real-time Biomolecular Interaction Analysis (BIA) (see, e.g., Sjolander, S. and Urbaniczky, C, 1991, Anal. Chem. 63:2338 2345 and Szabo et al, 1995, Curr. Opin. Struct. Biol. 5:699 705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. In differential centrifugation, marker/probe complexes may be separated from uncomplexed assay components through a series of centrifugal steps, due to the different sedimentation equilibria of complexes based on their different sizes and densities (see, for example, Rivas and Minton (1993) Trends Biochem. Sci. 18:284). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the marker/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard (1998) J. Mol. Recognit. 11:141; Hage and Tweed (1997) J. Chromatogr. B. Biomed. Sci. Appl. 12:499). Gel electrophoresis may also be employed to separate complexed assay components from unbound components (see, e.g., Ausubel et al, ed., Current Protocols in Molecular Biology, John Wiley & Sons, New York, 1987 1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In certain exemplary embodiments, the level of mRNA corresponding to the marker can be determined either by in situ and/or by in vitro formats in a biological sample using methods known in the art. Many expression detection methods use isolated RNA. For in vitro methods, any RNA isolation technique that does not select against the isolation of mRNA can be utilized for the purification of RNA from blood cells (see, e.g., Ausubel et al, ed., Current Protocols in Molecular Biology, John Wiley & Sons, New York 1987 1999). Additionally, large numbers of cells and/or samples can readily be processed using techniques well known to those of skill in the art, such as, for example, the single-step RNA isolation process of Chomczynski (1989, U.S. Pat. No. 4,843,155).

Isolated mRNA can be used in hybridization or amplification assays that include, but are not limited to, Southern or Northern analyses, polymerase chain reaction analyses and probe arrays. In certain exemplary embodiments, a diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in a gene chip array. A skilled artisan can readily adapt known mRNA detection methods for use in detecting the level of mRNA encoded by the markers of the present invention.

An alternative method for determining the level of mRNA corresponding to a marker of the present invention in a sample involves the process of nucleic acid amplification, e.g., by RT-PCR (the experimental embodiment set forth in U.S. Pat. Nos. 4,683,195 and 4,683,202), COLD-PCR (Li et al. (2008) Nat. Med. 14:579), ligase chain reaction (Barany, 1991, Proc. Natl. Acad. Sci. USA, 88:189), self-sustained sequence replication (Guatelli et al., 1990, Proc. Natl. Acad. Sci. USA 87:1874), transcriptional amplification system (Kwoh et al. (1989) Proc. Natl. Acad. Sci. USA 86:1173), Q-Beta Replicase (Lizardi et al. (1988) Bio/Technology 6:1197), rolling circle replication (U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the sample (e.g., a bodily fluid (e.g., blood cells)) prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations may be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in a patient sample from one source to a patient sample from another source, e.g., to compare a population of phagocytic from an individual to a population of non-phagocytic cells from the individual.

In one embodiment of this invention, a protein or polypeptide corresponding to a marker is detected. In certain embodiments, an agent for detecting a protein or polypeptide can be an antibody capable of binding to the polypeptide, such as an antibody with a detectable label. As used herein, the term “labeled,” with regard to a probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. Antibodies can be polyclonal or monoclonal. An intact antibody, or a fragment thereof (e.g., Fab or F(ab′)2) can be used. In one format, antibodies, or antibody fragments, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well known supports or carriers include glass, polystyrene, polypropylene, polyethylene, dextran, nylon, amylases, natural and modified celluloses, polyacrylamides, gabbros, magnetite and the like.

A variety of formats can be employed to determine whether a sample contains a protein that binds to a given antibody. Examples of such formats include, but are not limited to, competitive and non-competitive immunoassay, enzyme immunoassay (EIA), radioimmunoassay (MA), antigen capture assays, two-antibody sandwich assays, Western blot analysis, enzyme linked immunosorbant assay (ELISA), a planar array, a colorimetric assay, a chemiluminescent assay, a fluorescent assay, and the like. Immunoassays, including radioimmmunoassays and enzyme-linked immunoassays, are useful in the methods of the present invention. A skilled artisan can readily adapt known protein/antibody detection methods for use in determining whether cells (e.g., bodily fluid cells such as blood cells) express a marker of the present invention.

One skilled in the art will know many other suitable carriers for binding antibody or antigen, and will be able to adapt such support for use with the present invention. For example, protein isolated from cells (e.g., bodily fluid cells such as blood cells) can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

In certain exemplary embodiments, assays are provided for diagnosis, prognosis, assessing the risk of developing prostate cancer, assessing the efficacy of a treatment, monitoring the progression or regression of prostate cancer, and identifying a compound capable of ameliorating or treating prostate cancer. An exemplary method for these methods involves obtaining a bodily fluid sample from a test subject, isolating phagocytes and non-phagocytes, and contacting the phagocytes and non-phagocytes with a compound or an agent capable of detecting one or more of the markers of the disease or condition, e.g., marker nucleic acid (e.g., mRNA, genomic DNA), marker peptide (e.g., polypeptide or protein), marker lipid (e.g., cholesterol), or marker metabolite (e.g., creatinine) such that the presence of the marker is detected. In one embodiment, an agent for detecting marker mRNA or genomic DNA is a labeled nucleic acid probe capable of hybridizing to marker mRNA or genomic DNA. The nucleic acid probe can be, for example, a full-length marker nucleic acid or a portion thereof. Other suitable probes for use in the diagnostic assays of the invention are described herein.

As used herein, a compound capable of ameliorating or treating prostate cancer can include, without limitations, any substance that can improve symptoms or prognosis, prevent progression of the prostate cancer, promote regression of the prostate cancer, or eliminate the prostate cancer.

The methods of the invention can also be used to detect genetic alterations in a marker gene, thereby determining if a subject with the altered gene is at risk for developing prostate cancer characterized by misregulation in a marker protein activity or nucleic acid expression. In certain embodiments, the methods include detecting, in phagocytes, the presence or absence of a genetic alteration characterized by an alteration affecting the integrity of a gene encoding a marker peptide and/or a marker gene. For example, such genetic alterations can be detected by ascertaining the existence of at least one of: 1) a deletion of one or more nucleotides from one or more marker genes; 2) an addition of one or more nucleotides to one or more marker genes; 3) a substitution of one or more nucleotides of one or more marker genes, 4) a chromosomal rearrangement of one or more marker genes; 5) an alteration in the level of a messenger RNA transcript of one or more marker genes; 6) aberrant modification of one or more marker genes, such as of the methylation pattern of the genomic DNA; 7) the presence of a non-wild type splicing pattern of a messenger RNA transcript of one or more marker genes; 8) a non-wild type level of a one or more marker proteins; 9) allelic loss of one or more marker genes; and 10) inappropriate post-translational modification of one or more marker proteins. As described herein, there are a large number of assays known in the art which can be used for detecting alterations in one or more marker genes.

In certain embodiments, detection of the alteration involves the use of a probe/primer in a polymerase chain reaction (PCR) (see, e.g., U.S. Pat. Nos. 4,683,195, 4,683,202 and 5,854,033), such as real-time PCR, COLD-PCR (Li et al. (2008) Nat. Med. 14:579), anchor PCR, recursive PCR or RACE PCR, or, alternatively, in a ligation chain reaction (LCR) (see, e.g., Landegran et al. (1988) Science 241:1077; Prodromou and Pearl (1992) Protein Eng. 5:827; and Nakazawa et al. (1994) Proc. Natl. Acad. Sci. USA 91:360), the latter of which can be particularly useful for detecting point mutations in a marker gene (see Abravaya et al. (1995) Nucleic Acids Res. 23:675). This method can include the steps of collecting a sample of cell free bodily fluid from a subject, isolating nucleic acid (e.g., genomic, mRNA or both) from the sample, contacting the nucleic acid sample with one or more primers which specifically hybridize to a marker gene under conditions such that hybridization and amplification of the marker gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self-sustained sequence replication (Guatelli et al., (1990) Proc. Natl. Acad. Sci. USA 87:1874), transcriptional amplification system (Kwoh et al., (1989) Proc. Natl. Acad. Sci. USA 86:1173), Q Beta Replicase (Lizardi et al. (1988) BioTechnology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in one or more marker genes from a sample can be identified by alterations in restriction enzyme cleavage patterns. For example, sample and control DNA is isolated, optionally amplified, digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in one or more of the markers described herein can be identified by hybridizing a sample and control nucleic acids, e.g., DNA or RNA, to high density arrays containing hundreds or thousands of oligonucleotides probes (Cronin et al. (1996) Human Mutation 7: 244; Kozal et al. (1996) Nature Medicine 2:753). For example, genetic mutations in a marker nucleic acid can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence a marker gene and detect mutations by comparing the sequence of the sample marker gene with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147).

Other methods for detecting mutations in a marker gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type marker sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with 51 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397; Saleeba et al. (1992) Methods Enzymol. 217:286. In one embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in marker cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657). According to an exemplary embodiment, a probe based on a marker sequence, e.g., a wild-type marker sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in marker genes. For example, single strand conformation polymorphism (SSCP) may be used to detect differences in electrophoretic mobility between mutant and wild type nucleic acids (Orita et al. (1989) Proc. Natl. Acad. Sci. USA 86:2766, see also Cotton (1993) Mutat. Res. 285:125; and Hayashi (1992) Genet. Anal. Tech. Appl. 9:73). Single-stranded DNA fragments of sample and control marker nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucl. Acids Res. 17:2437) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

An exemplary method for detecting the presence or absence of an analyte (e.g., DNA, RNA, protein, polypeptide, or the like) corresponding to a marker of the invention in a biological sample involves obtaining a bodily fluid sample (e.g., blood) from a test subject and contacting the bodily fluid sample with a compound or an agent capable of detecting one or more markers. Detection methods described herein can be used to detect one or more markers in a biological sample in vitro as well as in vivo. For example, in vitro techniques for detection of mRNA include Northern hybridizations and in situ hybridizations. In vitro techniques for detection of a polypeptide corresponding to a marker of the invention include enzyme linked immunosorbent assays (ELISAs), Western blots, immunoprecipitations and immunofluorescence. In vitro techniques for detection of genomic DNA include Southern hybridizations. Furthermore, in vivo techniques for detection of a polypeptide corresponding to a marker of the invention include introducing into a subject a labeled antibody directed against the polypeptide. For example, the antibody can be labeled with a radioactive marker whose presence and location in a subject can be detected by standard imaging techniques. Because each marker is also an analyte, any method described herein to detect the presence or absence of a marker can also be used to detect the presence or absence of an analyte.

The markers useful in the methods of the invention can include any mutation in any one of the markers. Mutation sites and sequences can be identified, for example, by databases or repositories of such information, e.g., The Human Gene Mutation Database (www.hgmd.cf.ac.uk), the Single Nucleotide Polymorphism Database (db SNP, www.ncbi.nlm.nih.gov/proj ects/SNP), and the Online Mendelian Inheritance in Man (OMIM) website (www.ncbi.nlm.nih.gov/omim).

The present invention also provides kits that comprise marker detection agents that detect at least one or more of the prostate cancer markers described herein.

The present invention also provides methods of treating or preventing prostate cancer in a subject comprising administering to said subject an agent that modulates the activity or expression or disrupts the function of at least one or more of the markers of this invention.

The one or more markers identified by this invention (e.g., markers in Table 1, Table 2, and/or Table 3) may be used in the treatment of prostate cancer. For example, a marker (e.g., a protein or gene) identified by the present invention may be used as a molecular target for a therapeutic agent. A marker identified by the invention also may be used in any of the other methods of the invention, e.g., for monitoring the progression or regression of a disease or condition. In certain embodiments, the one or more markers identified by the methods of this invention may have therapeutic potential. For example, if a marker is identified as being up-regulated (or down-regulated), or activated (or inhibited) in phagocytic cells from a subject having prostate cancer, a compound or an agent that is capable of down-regulating (or up-regulating) or inhibiting (or activating) said marker may be useful in treating prostate cancer. Similarly, a gene protein expression level, a protein expression level, or a combination thereof may be useful in this aspect of the invention.

In some embodiments, a kit may be provided with reagents to measure at least two of the panel of biomarkers. The panel of biomarkers to be measured with the kit may include two or more biomarkers from the markers listed in Table 1, Table 2, and/or Table 3. The kit may include reagents to measure a panel of biomarkers that includes two, three, four, five, six, seven or more biomarkers combined together to measure a subject's biomarker panel. The kit may be provided with one or more assays provided together in a kit. By way of non-limiting example, the kit may include reagents to measure the biomarkers in one assay. In some embodiments, the kit may include reagents to measure the biomarkers in more than one assay. Some kits may include a 4-plex assay and a 2-plex assay while other kits may include different combinations of assays to cover all the biomarkers needed to be measured. The kit may also include reagents to measure a biomarker individually and other biomarkers in a 2-, 4-, or 8-plex assay. Any combination of reagents and assay may be combined in a kit to cover all the biomarkers needed.

Unless otherwise defined herein, scientific and technical terms used in this application shall have the meanings that are commonly understood by those of ordinary skill in the art. Generally, nomenclature used in connection with, and techniques of, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

All of the above, and any other publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The following examples are set forth as being representative of the present invention. These examples are not to be construed as limiting the scope of the invention as these and other equivalent embodiments will be apparent in view of the present disclosure and accompanying claims.

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein. All publications, patents, and patent applications cited herein are hereby incorporated by reference in their entirety for all purposes.

### EXAMPLES

The following examples illustrate but do not limit the compounds, compositions, and methods of the present invention. Other suitable modifications and adaptations of the variety of conditions and parameters normally encountered in clinical therapy and which are obvious to those skilled in the art are within the spirit and scope of the invention.

### Example 1

Discovery and characterization of prostate cancer signatures

One thousand and eighteen patients were enrolled in the study. The inclusion and exclusion criteria for the study was as follows: Inclusion criteria: subject willing and able to provide the following: informed consent; approximately 40 cc of blood (30 cc for SNEP assay, 10 cc for plasma analysis); pre-biopsy blood draw from male patient determined by physician to have a risk profile warranting a prostate biopsy; and post-biopsy blood draw from male patient that had a biopsy greater than 30 days prior but less than 1 year of study entry and/or had not undergone definitive therapy. Subjects were excluded from the study if: age less than 50 years old; any known concurrent cancer (except non-melanoma skin cancer) or any history of cancer in the last 5 years); any form of androgen deprivation therapy (ADT) except 5-alpha-reductase inhibitors.

Approximately 40 cc of blood was collected from each patient into blood cell preparation tubes and serum separation tubes (SST). Time of blood draws were recorded. Approximately 4 ml of blood was drawn into an EDTA blood collection tube, 4 ml of blood drawn into a serum separation tube, and 8 ml of blood was drawn into each of three separate blood collection tubes. All tubes were inverted approximately 8-10 times. The EDTA tube was kept at 4° C. until transported to lab for analysis.

Blood samples drawn into the EDTA tubes was used for plasma collection. When the EDTA tube arrived into the laboratory, the tube was spun at 300×g for 10 minutes, the plasma was drawn off the top being careful not to disrupt the buffy coat and transferred to a new 15 mL conical tube. The 15 mL conical tube was spun at max speed for 10 minutes and the plasma was removed off the top and aliquot into EDTA plasma tubes 1.5 ml screw cap tubes and frozen at −80° C., with the time/date of the plasma extraction recorded.

The serum separation tube was spun according to standard protocol for prostate-specific antigen (PSA) preparation (see, e.g., Oesterling et al., JAMA 1993 Aug. 18; 270:860-864; Smith et al., CA Cancer J Clin 2002; 52:8-22; Blute et al., J Urol 2001 January; 165(1):119-125). The blood collection tubes were centrifuged for 20 minutes at 1760×g. After centrifugation RBCs were separated from Peripheral Blood Mononuclear Cells (PBMCs). After centrifugation tubes were inverted 8-10 times and placed at 4° C. All tubes were transported the same day or overnight to the lab at 4° C. Upon arrival to the lab, complete blood counts and PSA analysis was performed. Tubes were processed within 72 hours of the blood draw.

Peripheral blood mononuclear cells were isolated from the blood samples of each patient. Two 30 μl aliquots of PBMCs were taken and used as unseparated controls on the flow cytometer and a 2 ml sample of PBMCs was used as an unseparated sample. The remaining PBMCs were split for cell separation. The volume was determined and ⅓ used for isolation of T cells (CD2+ cells) and ⅔ used for isolation of monocytes (CD14+ cells).

PBMCs were centrifuged at 300×g for 10 min to pellet cells. The supernatant was removed and cells re-suspended in buffer (225 μl for CD2 and 400 μl for CD14). Magnetic beads specific to either CD2 or CD14 were added to the re-suspended cells, respectively (25 μl for CD2 and 100 μl for CD14). Beads were incubated with cells for 15 minutes at 4° C. After incubation, 250 μl of buffer was added to the CD2 sample to bring the total volume to 500 μl.

Each sample was placed into a separate well of a 24 well column block and cells were separated on a MultiMACS™ Ce1124 Separator Plus (Miltenyi Biotec) using positive selection for the cells attached to magnetic beads. Separated cells were eluted into a 24 well plate using a vacuum chamber. The 24 well plate containing CD2 and CD14 cells was removed from the vacuum chamber. Two 15 μl aliquots of separated cells were taken from each well for use on a flow cytometer in order to assess and verify sample purity using a MACS Quant Flow cytometer. A 2 ml aliquot of unseparated PBMCs was centrifuged at 300×g for 10 minutes. The supernatant was removed and the cell pellet re-suspended in 500 μl of buffer. The sample was added to the empty wells of a 24 well plate. RNA extraction from the samples in the 24 well plate was performed using the Thermo Scientific™ KingFisher™ Flex Purification System. RNA extraction was paused after the first wash and samples were transferred to a 96 well plate before extraction continued.

After extraction was complete, RNA was transferred to 1.5 ml tubes and the quantity and purity of the RNA samples were determined. RNA integrity was assessed using the Agilent TapeStation. An RNA integrity number (RIN) value ≥7 was achieved for each sample to proceed with the library preparation. RNA sequencing libraries were generated using the Illumina TruSeq Targeted RNA Custom Kit following the manufacturer's user guide. Completed libraries were quantitated using QPCR and the size of the fragments visualized using the Agilent Bioanalyzer.

Libraries were combined in equimolar proportions into a single pool. The pool was loaded onto one lane of a flow cell for clustering. The flow cell was run on a 50 bp single read sequencing run on the Illumina HiSeq2500. Onboard image processing and base calling was performed. The sequence data quality score (Q score) was used as a quality control metric with the specification that ≥80% of bases must have a Q score of ≥30 (The quality or Q score measures the probability that a base is called incorrectly. A Q score of 30 reflects that the probability of an incorrect base call is 1 in 1000 for an inferred base call accuracy rate of 99.9%).

RNA sequencing data for T cells, macrophages, and monocytes (isolated using anti-CD2 and anti-CD14 antibodies described above, N=1018 subjects) were aligned to transcriptome. Counts for approximately 25,000 genes were quantified and then normalized individually by cell type, to account for subject-wise library differences. Once normalized, gene expression ratios between CD2 and CD14 cells were calculated, in log-domain, for each subject.

Side clinical covariates for each subject included: age (in years since sample collection), race (as multiple binary variables: white, African American, Hispanic and Middle Eastern), Digital Rectal Exam (DRE, where binary, normal vs. normal is being considered), prostate volume (in log-domain) and total PSA (in log-domain).

The weighted sum of gene sequencing/expression ratios and clinical covariates were concatenated and used as the input to a sparse rank regression model to generate receiver operating characteristic curves. Analysis generated during development of embodiments of the invention generated prostate cancer Aggressiveness Index (PCAI) that aggregated maximum Gleason grade, number of positive biopsied cores (“cores positive”), and maximum involvement among the biopsied cores (e.g., that provided the ability to discriminate, using one or more signatures identified herein, between aggressiveness or indolence of cancer (e.g., prostate cancer) in a subject). The PCAI model is a prostate biopsy summary on an ordinal scale (0-4, 0 being negative biopsy and 4 being very aggressive cancer), that aggregates maximum Gleason grade, number of positive biopsied cores and the maximum involvement among the biopsied cores: a Score of 0 meant no evidence of cancer on 12 core or more biopsy; a Score of/meant low grade⁺ and low volume⁺ (i.e., Grade 1, 1-2 cores up to 10%; or Grade 2, 1-2 cores up to 5%); a Score of 2 meant low grade⁺⁺ and low volume (i.e., Grade 1, 3-5 cores [20-40%]; or Grade 2, 3-4 cores [10-20%]; or Grade 3, 1-2 cores [1-5%]); a Score of 3 meant intermediate grade and intermediate volume (i.e., Grade 1, 6-12 cores [50-100%]; or Grade 2, 5-9 cores [30-70%]; or Grade 3, 3-6 cores [10-50%]; or Grade 4, 1-2 cores [1-5%]; or Grade 5, 1 core [1-2%]); and a Score of 4 meant high grade and high volume (i.e., Grade 2-3, >5 cores [>50%]; or Grade 4, >2 cores [>10%]; or Grade 5, >1 core [>1%]).

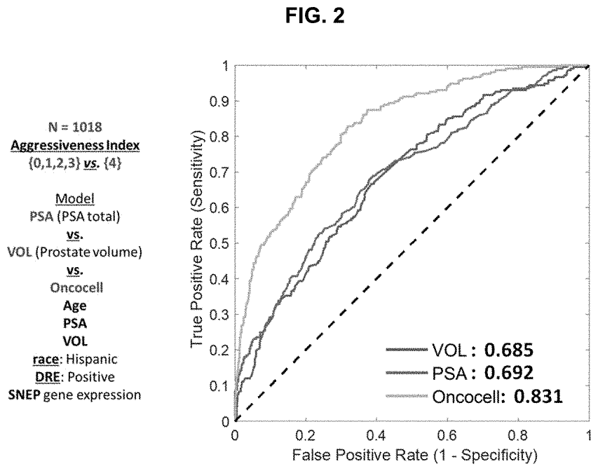

A total of 1018 subjects were analyzed. The parameters of the model (model coefficients, one per input) were estimated on a subset of the first N=713 (70%) subjects enrolled in the study (See FIG. 2). The model also estimated which of its inputs were predictive of the output, i.e., the PCAI. Inputs with predictive value were assigned a nonzero model coefficient (weight). The subset of inputs (genes and clinical covariates) identified by the model as predictive, termed “PC signature”, were solely responsible for the predictions made by the model, inputs not in the signature (with zero model coefficients), were ignored. After training the model on 713/1018 patients, 61 covariates were identified having non-zero weights (See Table 1).

TABLE 1
Exemplary Prostate Cancer Genomic & Clinical Covariates Identified

| GENOMIC | CLINICAL |
|---|---|
| 1: ANGPT4 | 1: Age |
| 2: BAMBI | 2: DRE_Yes |
| 3: C2orf27B | Abnormal |
| 4: C3orf67 | Positive |
| 5: C9orf135 | 3: PSA_log |
| 6: CA2 | 4: Race_hispanic |
| 7: CLCNKA | 5: Vol_log |
| 8: COCH | |
| 9: COLEC11 | |
| 10: CYGB | |
| 11: DSP | |
| 12: EGFL6 | |
| 13: EGR2 | |
| 14: FCER1A | |
| 15: FLJ40194 | |
| 16: FST | |
| 17: FSTL1 | |
| 18: FTCD | |
| 19: GATA2 | |
| 20: GRID1 | |
| 21: HDGFRP3 | |
| 22: HIST1H2BG | |
| 23: HIST1H2BN | |
| 24: HOXA5 | |
| 25: ISLR2 | |
| 26: ITGA2B | |
| 27: KIR2DL4 | |
| 28: KLF17 | |
| 29: KRTAP5-8 | |
| 30: KRTAP5-9 | |
| 31: LOC100506462 | |
| 32: LOC729156 | |
| 33: MGC14436 | |
| 34: MIR1249 | |
| 35: MYL9 | |
| 36: MYO1D | |
| 37: NPBWR1 | |
| 38: OOEP | |
| 39: PDZK1IP1 | |
| 40: PKHD1L1 | |
| 41: PPBP | |
| 42: RAB6B | |
| 43: ROR2 | |
| 44: RSPH9 | |
| 45: SLC4A9 | |
| 46: SNORD42A | |
| 47: SNORD49B | |
| 48: SPG20OS | |
| 49: ST6GALNAC2 | |
| 50: TAGLN3 | |
| 51: tAKR | |
| 52: TEKT5 | |
| 53: TMEM133 | |
| 54: TRPM1 | |
| 55: WNT9A | |
| 56: ZNF474 | |

The performance characteristics of the model, in terms of Area Under the Receiving Operating Characteristic (AUROC), were evaluated on the remaining, chronologically ordered 305 (30%) patients, not used for model estimation, in order to obtain unbiased estimates of model performance (See FIG. 1, Validation Set).

One of the PC signatures identified contained multiple inputs, for example, clinical covariates including age, DRE, prostate volume, and total PSA, as well as biomarker covariates including those listed in Table 2.

TABLE 2
Exemplary Prostate Cancer Genomic & Clinical Covariates Identified

| GENOMIC | CLINICAL |
|---|---|
| 1: BAMBI | 1: Age |
| 2: C3orf67 | 2: DRE_Yes |
| 3: C9orf135 | Abnormal |
| 4: COCH | Positive |
| 5: FLJ40194 | 3: PSA_log |
| 6: FST | 4: Race_hispanic |
| 7: FSTL1 | 5: Vol_log |
| 8: GATA2 | |
| 9: HDGFRP3 | |
| 10: MYO1D | |
| 11: OOEP | |
| 12: SNORD42A | |
| 13: tAKR | |
| 14: TMEM133 | |
| 15: WNT9A | |

Other PC gene signature markers and clinical covariates that may be measured in accordance with the present disclosure are set forth in Table 3.

TABLE 3
Exemplary Prostate Cancer Genomic & Clinical Covariates Identified

| GENOMIC | CLINICAL |
|---|---|
| 1: C11orf94 | 1: Age |
| 2: C9orf135 | 2: DRE_Yes |
| 3: DSP | Abnormal |
| 4: EGFL6 | Positive |
| 5: FST | 3: PSA_log |
| 6: FSTL1 | 4: Race_hispanic |
| 7: GATA2 | 5: Vol_log |
| 8: GRID1 | |
| 9: KLF17 | |
| 10: KRTAP5-8 | |
| 11: MID1 | |
| 12: MYO1D | |
| 13: OOEP | |
| 14: RSPH9 | |
| 15: TAGLN3 | |

In order to further validate and confirm the usefulness of a SNEP assay using a PC signature described herein, ROC curves using SNEP utilizing a PC signature comprising the biomarkers of Table 3 together with clinical covariates age, DRE, prostate volume, and total PSA (see FIG. 2, Oncocell) were compared against the ROC curves utilizing either prostate specific antigen (PSA) levels (see FIG. 2, PSA) or prostate volume (see FIG. 2, VOL). The ROC curves are shown in FIG. 2.

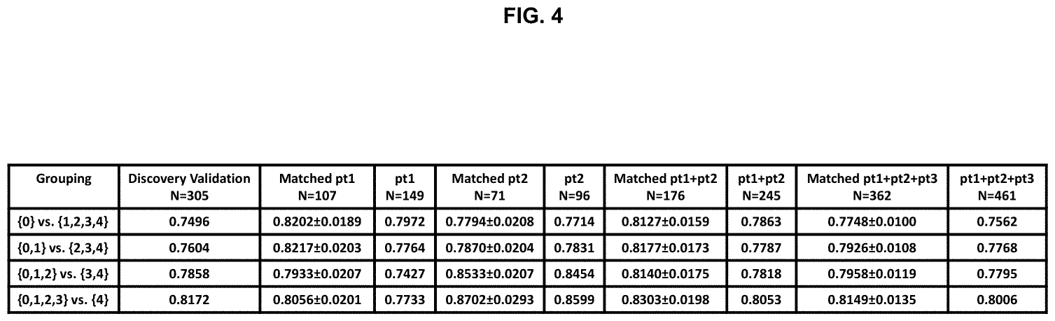

Data generated utilizing SNEP and a prostate cancer signature comprising the biomarkers of Table 3 together with clinical covariates age, DRE, prostate volume, and total PSA is shown in FIG. 4.

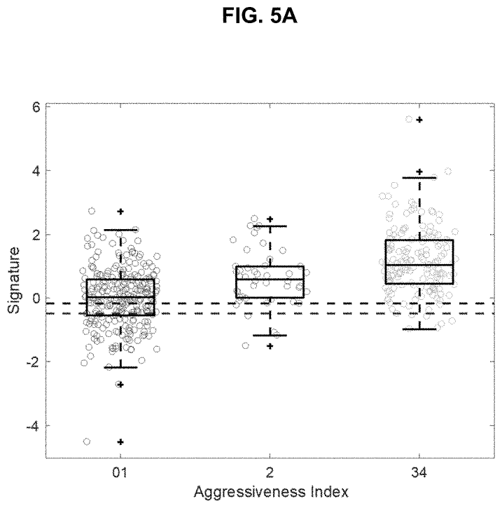

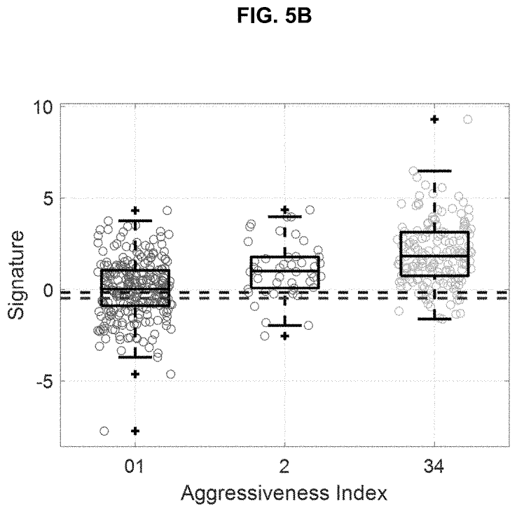

FIG. 5 shows patient scoring on the prostate cancer aggressiveness index according to one embodiment of the invention using A) nineteen covariates shown in FIG. 4, or B) using the same covariates minus DRE.

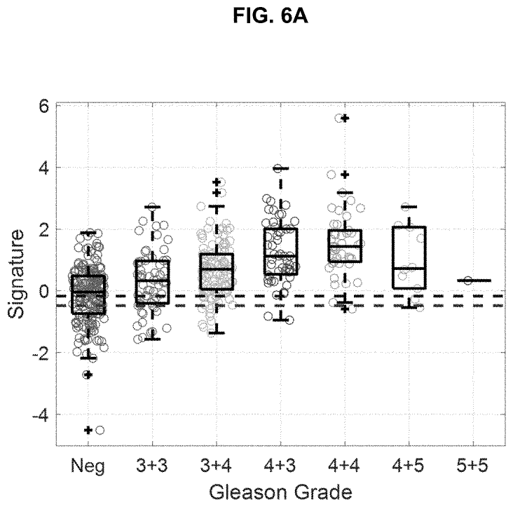

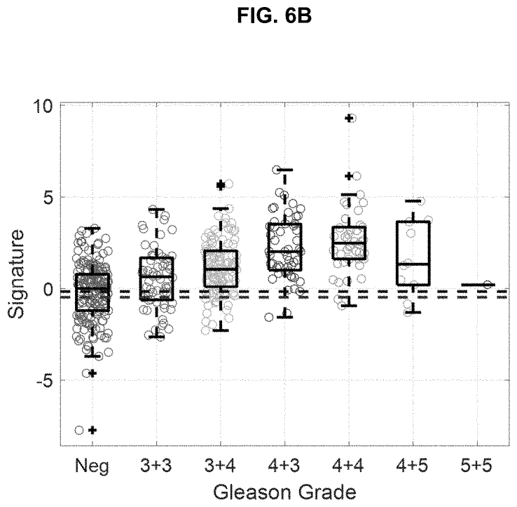

FIG. 6 shows patient scoring on the prostate cancer aggressiveness index according to one embodiment of the invention compared to Gleason scoring using A) nineteen covariates shown in FIG. 4, or B) using the same covariates minus DRE.

Multiple PC signatures can be generated using this approach. For example, given the dataset of N=713 subjects, more than one combination of inputs (PC signatures, see, e.g., Table 1, Table 2, and/or Table 3) that yield comparatively similar performance metrics (statistically indifferent given the sample size) were made possible. Further, other inputs that correlate substantially with any of the elements of the signature may be potentially added to a modified, larger, signature without significantly impacting the performance characteristics of the model with the larger signature relative to the original.

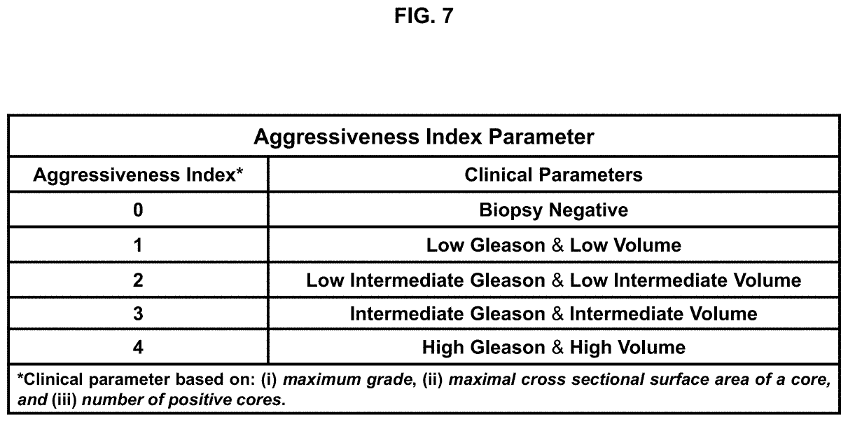

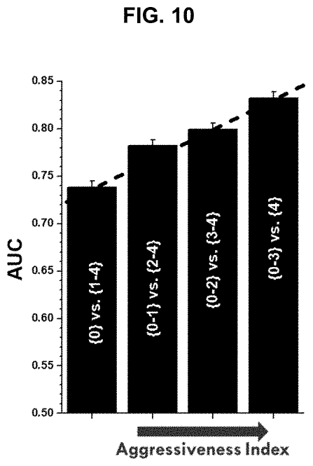

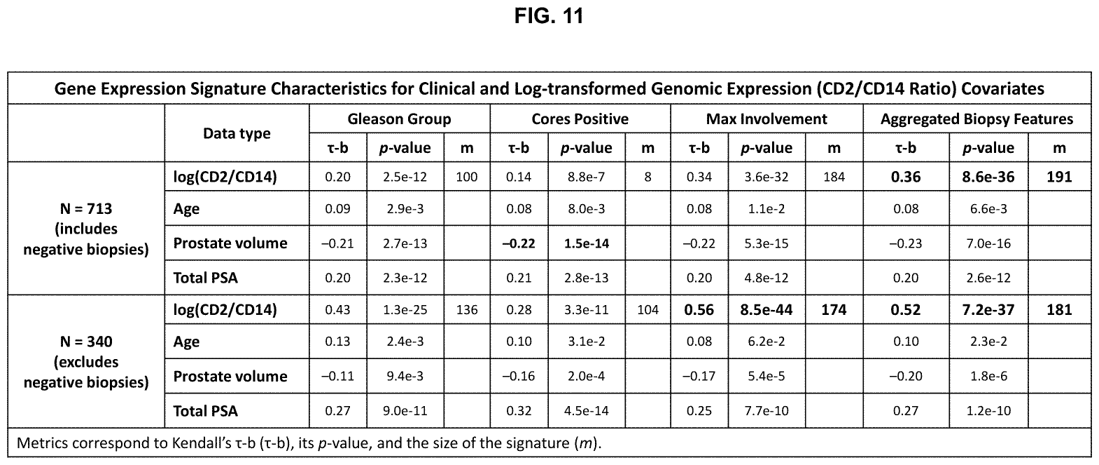

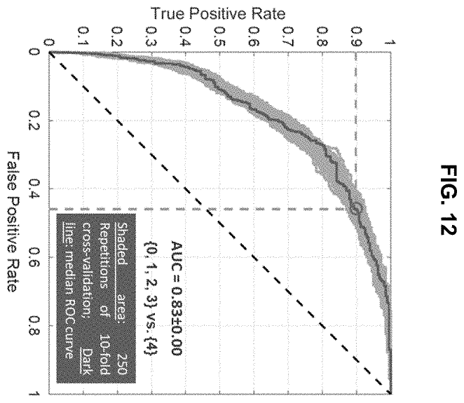

Thus, the Aggressiveness Index incorporates 3 endpoints: 1) Gleason Grade (GG), 2) number of Cores Positive (CP), and 3) Maximum Involvement (MI). After training this model on the 713 patients, a genomic signature was identified that is predictive of GG, CP, MI, and AI (see FIG. 7-FIG. 10). The signature scores (calculated as the average of the positively associated transcripts minus the negatively associated transcripts) were significantly associated with the four endpoints. Gene expression signature characteristics for the tested patients are shown in FIG. 11. A total of 61 covariates were identified and used to generate a ROC curve. For these patients, the following performance estimates were obtained: AUC: 0.83±0.01; TPR: 0.90±0.00; FNR: 0.10±0.00; and NPV: 0.95±0.01) (see FIG. 12).

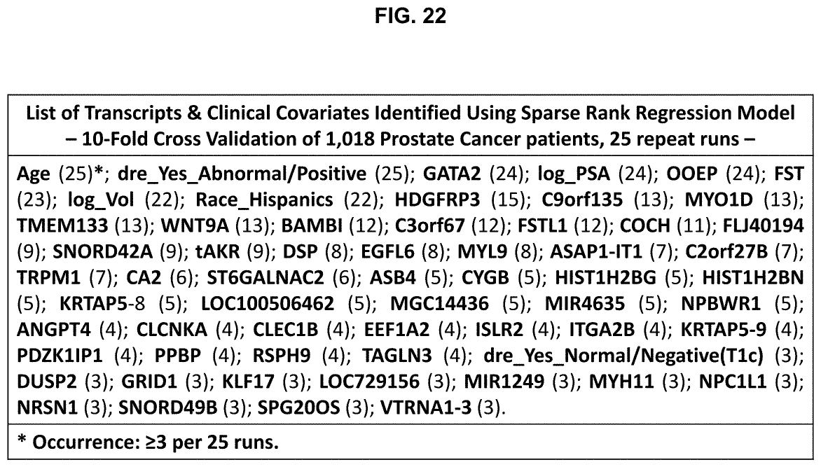

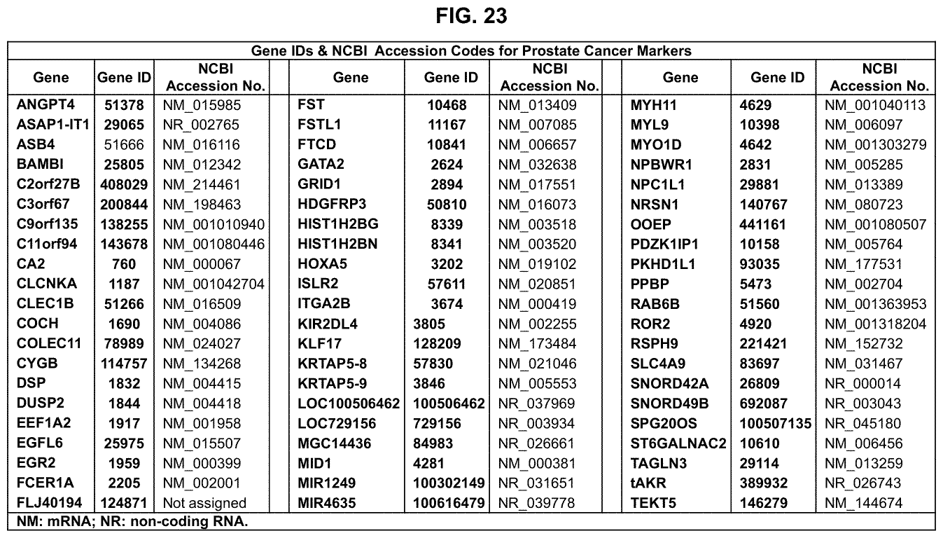

FIG. 22 provides a listing of 54 markers and 6 clinical covariates identified in prostate cancer patients when a Sparse Rank Regression Model was run 25 times. FIG. 23 provides a listing of PC covariates (including National Center For Biotechnology Information (NCBI) accession numbers and gene ID numbers available via the internet from the National Center For Biotechnology Information) that may be measured in accordance with the present disclosure.

### Example 2

Validation of the PCAI

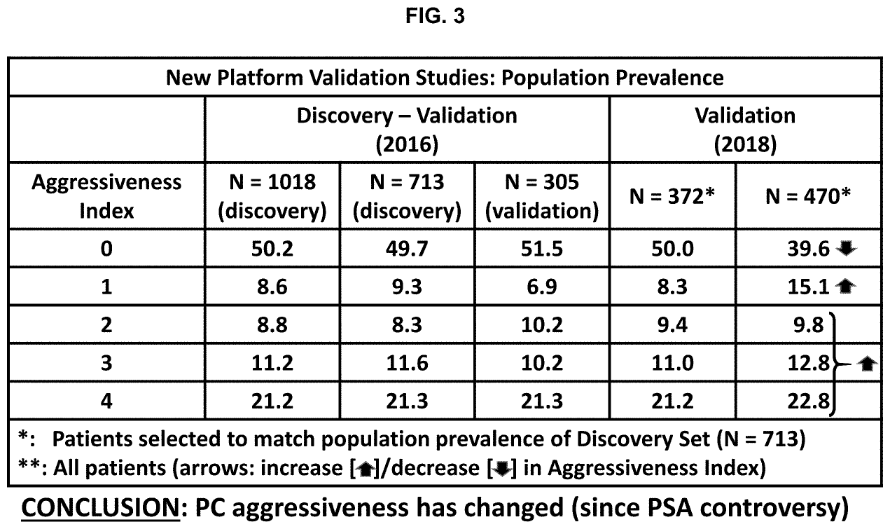

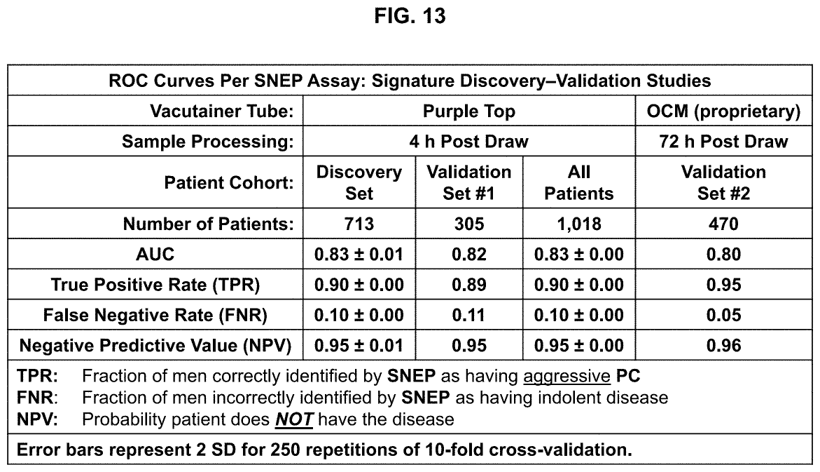

An independent, prospectively enrolled, cohort of N=470 new subjects were used to validate the findings of the discovery of the signatures identified in Example 1, namely, the model (defined by the signature and model coefficients) and its performance characteristics. RNA sequencing and clinical data for the validation cohort were processed following the same procedure as in Example 1, except that blood was collected in “OCM” tubes, which maintain the integrity of the RNA for at least 72 hours. Due to differences in the composition of the cohort in terms aggressiveness index proportions (prevalence) relative to the discovery cohort, a matched subset of N=372 subjects matched by aggressiveness index was down-selected from the complete N=470 subject cohort (See FIG. 3). Finally, the model was used to make predictions for the N=372 subjects and performance characteristics were evaluated following the same procedure as in the discovery phase. The results of the validation study are shown in FIG. 13. No significant differences were found between the performance characteristics of both phases of the study, therefore, the model and its performance characteristics were deemed statistically validated.

### Example 3

This example further demonstrates that real-time surveillance of gene expression in phagocytic and non-phagocytic white blood cells (WBCs)—via RNA sequencing of monocytes and lymphocytes obtained from a patient—enables the detection of immune-response signal changes. Such immune response signal changes are caused by (i) intrinsic inter-individual variability, e.g., gender, genetic/ethnic background, etc. (Whitney et al., Proc. Natl. Acad. Sci. USA, 100: 1896-901 (2003); Radich et al., Genomics, 83: 980-8 (2004); Cheung V G and Spielman R S., Nat. Rev. Genet., 10: 595-604 (2009); Xu et al., PLoS One, 6: e26905-e15 (2011); Hughes et al., Genome Biology, 16: 54-71 (2015)), (ii) epigenetic age-related (temporal) variations (Christensen et al., PLoS Genetics, 5: e1000602-e14 (2009); Pal S and Tyler J K, Science Advances, 2: e1600584-e602 (2016); Klutstein et al., Cancer Res, 76: 3446-50 (2016); Gopalan et al., Genetics, 206: 1659-74 (2017)), (iii) extrinsic intra-individual extracellular “milieu” stimuli, e.g., food/drink intake immediately prior to blood draw, smoking, recent vaccination, etc. (Hughes et al., Genome Biology, 16: 54-71, 33-44 (2015)), (iv) specific disease that a blood test aims to detect, e.g., prostate cancer (Huen et al., Int J Cancer, 133: 373-82 (2013); Wallace et al., Carcinogenesis, 35: 2074-83 (2014)), lung cancer (Showe et al., Cancer Res, 69: 9202-10 (2009); Zander et al., Clin Cancer Res, 17: 3360-7 (2011); Kossenkov et al., PLoS ONE; 7: e34392-e9 (2012), and pancreatic cancer (Baine et al., Cancer Biomarkers: Section A of Disease Markers, 11: 1-14 (2011), and (v) other disease/conditions unrelated to the disease, e.g., arthritis (Batliwalla et al., Genes and Immunity, 6: 388-97 (2005)), acute infection (Ramilo et al., Blood, 109: 2066-77 (2007)), etc. that conventional blood tests aim not to detect.

Materials and Methods

Patient Population:

Blood samples were collected from 713 men visiting a urologist and suspected of having prostate cancer or known to have untreated prostate cancer, all with available prostate needle biopsy data obtained within one year.

Inclusion Criteria:

Men were eligible for enrollment in the study if they (i) were determined by their physician to have a risk profile that warranted a prostate biopsy, (ii) had a biopsy greater than 90 days but less than 1 year prior to study entry and had not undergone definitive therapy, and/or (iii) were on active surveillance such that a biopsy would be done within the next year.

Exclusion Criteria:

Men were not eligible for enrollment in this study if they 1) were less than 40 or greater than 75 years old, 2) had any known concurrent cancer except non-melanoma skin cancer, or any history of cancer in the last 5 years, and 3) had any form of androgen deprivation therapy (ADT), with the exception of 5 alpha reductase inhibitors.

Clinical and Pathological Data Abstraction:

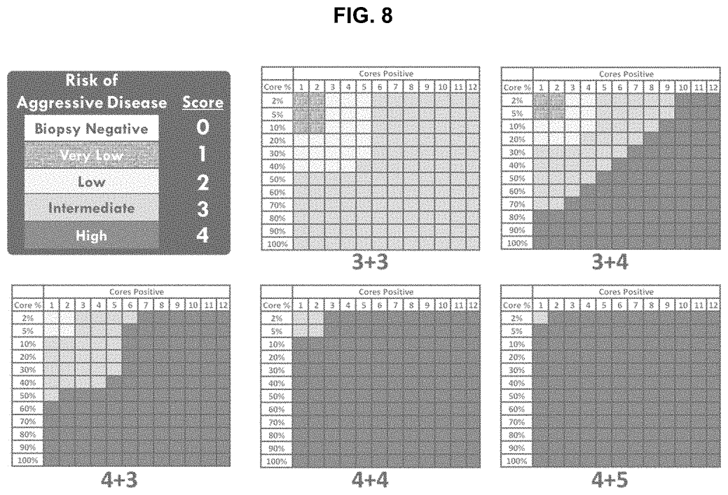

Clinical, laboratory, and pathology data of each patient was abstracted from the electronic medical record (EMR) charts and entered into an Electronic Data Capture (EDC) system by the research departments at the various study institutions. Pathologists at all three institutions agreed on the main standard data points to be included in the needle biopsy pathology reports. The current International Society of Urological Pathology (ISUP) modified Gleason grading system was used (Egevad et al., APMIS, 124: 433-5 (2016)) and the data from the highest-grade group of a single core was recorded. The maximal cross sectional surface area of tumor on a single core and the number of positive cores were recorded in the EDC. An aggregate that is based on highest Gleason grade group, number of positive cores, and maximal percent cross sectional surface area involvement of tumor was produced such that negative biopsies were a 0, low grade-small volume tumors were a 1, and high volume-high grade tumors was a 4. The specifics of the aggregated biopsy data are presented in FIG. 8.

Sample Collection and Transport Conditions:

Blood samples were obtained from three large urology practices: Comprehensive Urology (Detroit, Mich.), Michigan Institute of Urology (Detroit, Mich.), and Urology Austin (Austin, Tex.). All the enrolled patients signed written informed consent forms per ethical guidelines of the Institutional Review Board. Blood samples were collected in four K2EDTA BD VACUTAINER™ tubes (Cat. No. 366643, BD Biosciences, San Jose, Calif.) and transferred to the processing location on ice at 4° C. and processed 4 hours after draw time.

CD2/CD14 Cell Separation:

Blood was pooled from three blood tubes at 4° C. The blood was split into ⅓ and ⅔ aliquots for CD2 and CD14 cell type isolations, respectively. Specially formulated positive selection MACS Microbeads using anti-CD2 antibodies and anti-CD14 antibodies (Cat. No. 130-101-329 and 130-101-328, respectively, Miltenyi Biotech, Bergisch Gladbach, Germany) were added to the aliquots of blood at a volume of 25 μl CD2 beads per 1 ml blood and 50 μl CD14 beads per 1 ml blood. Beads were incubated with the blood samples for 10 minutes at 4° C. The blood-bead suspensions were then processed at 4° C. using a positive selection template on the autoMACS Pro Separator (Miltenyi Biotech) to isolate the CD2 and CD14 cells. Small aliquots of the isolated CD2 and CD14 cells were removed for flow cytometry analysis, while the remaining cells were pelleted by a 10-minute centrifugation at 300×g at 4° C. Following centrifugation, the supernatant was removed, 700 μL of room temperature QIAzol Lysis Reagent (Cat. No. 79306, Qiagen, Hilden, Germany) was added to each cell pellet, and the cell suspension pipetted up and down for 2 minutes to lyse the cells. The suspension was then vortexed for 1 minute to further homogenize the cell lysates and frozen at −80° C.

Flow Cytometry:

Following their isolation, aliquots of the two WBC populations were stained with 1) a positive dye mix containing human CD2-FITC, human CD36-APC-Vio770, and human MC CD14 Monocyte Cocktail for staining CD2 and CD14 cells, respectively, and 2) a negative dye mix consisting of human CD45-VioBlue, mouse IgG2b-FITC, mouse IgG2a-PE, mouse IgM-APC, and mouse IgG2a-APC-Vio770 (Miltenyi Biotech). Only samples with purity of ≥90% CD2 and CD14 were used in these studies.

RNA Extraction:

RNA extraction was accomplished using the miRNeasy Mini Kit (Cat. No. 217004, Qiagen). In essence, the frozen CD2 and CD14 cell samples (−80° C.) were thawed in a 37° C. dry bath (˜2.5 minutes) and incubated at room temperature (RT) for five minutes prior to the addition of 140 μL of chloroform and shaken vigorously for 15 seconds. Following a three minute RT incubation, the samples were centrifuged at 12,000×g (4° C., 15 min). The upper clear aqueous phase (˜350 μL) was transferred to a 2 mL collection tube that was then placed inside the QlAcube (Cat. No. 9001292, Qiagen), and poly(A) RNA was extracted using the miRNeasy Mini Kit per manufacturer's protocol. The quality and quantity of each RNA sample was determined on a Bioanalyzer 2100 (Agilent Technologies, Santa Clara, Calif.). Finally, the RNA samples were frozen at −80° C. and shipped to the Yale Center for Genome Analysis (YCGA) (West Haven, Conn.) for RNA sequencing. Only samples with high purity (RNA Integrity Number, RIN ≥9) were sent for sequencing.

Whole Genome RNA Sequencing

RNA Seq Library Prep:

mRNA was purified from approximately 200 ng of total RNA with oligo-dT beads and sheared by incubation at 94° C. Following first-strand synthesis with random primers, second strand synthesis was performed with dUTP for generating strand-specific sequencing libraries. The cDNA library was then end-repaired, A-tailed, the adapters were ligated, and second-strand digestion was performed by Uracil-DNA-Glycosylase. Indexed libraries that met appropriate cut-offs for both were then quantified by qRT-PCR using a commercially available kit (KAPA Biosystems) and insert size distribution was determined with the LabChip GX or Agilent Bioanalyzer. Samples with a yield of ≥0.5 ng/μL were sent for sequencing.

Flow Cell Preparation and Sequencing:

Sample concentrations were normalized to 10 nM and loaded onto Illumina Rapid or High-output flow cells at a concentration that yields 130-250 million passing filter clusters per lane. Samples were sequenced using 75 bp paired-end sequencing on an Illumina HiSeq 2500 according to Illumina's protocols. The 6 bp index was read during an additional sequencing read that automatically followed the completion of read 1. Data generated during sequencing runs were simultaneously transferred to the YCGA high-performance computing cluster. A positive control (prepared bacteriophage Phi X library) provided by Illumina was spiked into every lane at a concentration of 0.3% to monitor sequencing quality in real time.

Data Processing:

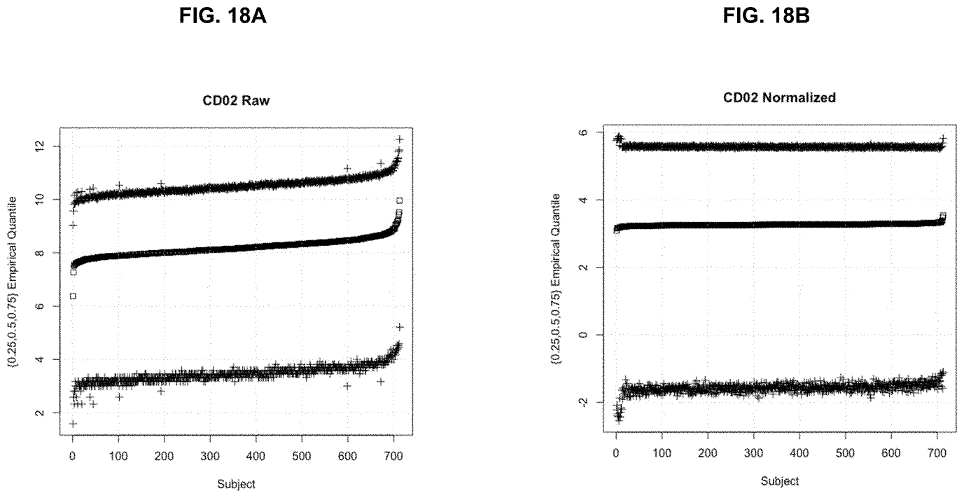

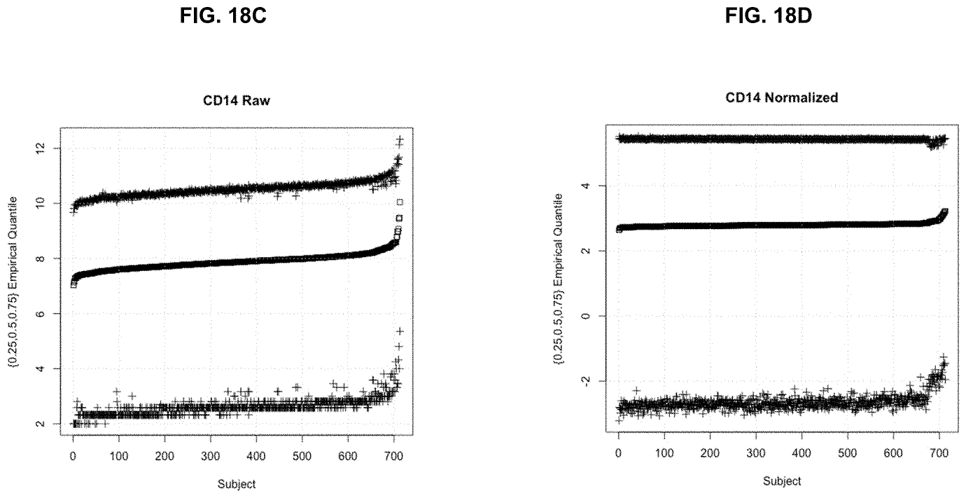

Signal intensities were converted to individual base calls during a run using the system's Real Time Analysis (RTA) software. Sample demultiplexing was performed using Illumina's CASAVA 1.8.2 software suite. Only data with a sample error rate less than 2% and a distribution of reads per sample in a lane that is within reasonable tolerance was used. Demultiplexed raw (FASTQ) RNA sequencing (RNA-seq) data was processed using (1) Trimmomatic (Bolger et al., Bioinformatics, 30: 2114-20 (2014)) for trimming, Bowtie2 (Langmead B and Salzberg S L., Nature Methods, 9: 357-9 (2012)) for alignment to UCSC (University of California, Santa Cruz) hg19 transcriptome, and Express (Roberts A and Pachter L., Nature Methods, 10: 71-3 (2012)) for quantification. Processed reads yielded counts for 23,368 transcripts (gene symbols) across 1,426 RNA samples (N=713 subjects), corresponding to 29.8±7.53M (Million) and 33.9±7.45M mapped reads from CD2 and CD14 samples, respectively (see FIG. 17 for the distributions of mapped reads). Initial filtering of the data resulted in a reduced set of 18,703 transcripts with observed expression (nonzero counts) in at least 15% of the samples in either CD2 or CD14. Sample normalization to account for RNA concentration differences was performed using Trimmed Mean M-Value (TMM) normalization (Robinson M D and Oshlack A, Genome Biology, 11: R25-R33 (2010)) (see FIGS. 18A-D for a summary of the samples distribution before and after normalization). To further identify transcripts with quantifiable expression changes between samples from different cell types, differential expression analysis was performed using a linear model and cell type as endpoint (dependent variable), which resulted in the final set of 10,643 transcripts with largest average CD2 to CD14 differences that were selected using <10% False Discovery Rate (FDR, Benjamini-Hochberg (50)) and >1.5 absolute fold change as thresholds.

Statistical Methods

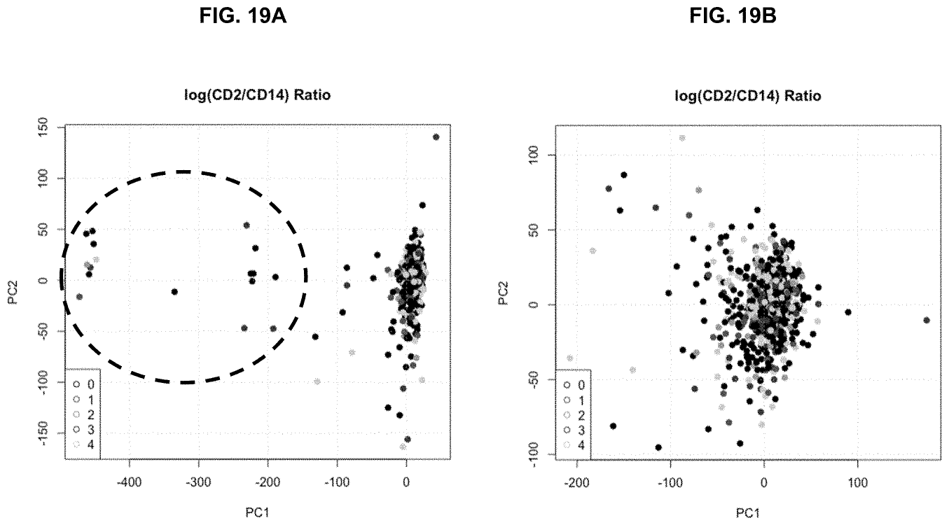

Exploratory analysis on log-transformed ratios of CD2 and CD14 data, log(CD2/CD14), revealed no statistically significant variation (p>0.05) due to sample quality metrics (e.g., RNA concentration, purity, and RNA integrity number); however, it identified 17 men (10 negative biopsies, 1 GG 1, 1 GG 2, 4 GG 3, and 1 GG 4) with outlying gene expression profiles when projecting the set of 10,643 transcripts into a one-dimensional principal subspace via Principal Component Analysis (PCA) (Jolliffe I., Principal Component Analysis, pp. 1094-6 (2011)). Outliers were defined as subjects whose absolute first Principal Component (PC1) representation was greater than 3×SD (standard deviation) away from the mean of PC1 (see FIGS. 19A AND 19B). For context, the first two principal components, PC1 and PC2, explained 31.7% and 5.5%, respectively, of the total variance in the data. After further examination, the 17 excluded subjects were verified as having lower and higher than average 0.25 quantiles on CD2 and CD14, respectively, which resulted in over dispersed log (CD2/CD14) distributions.

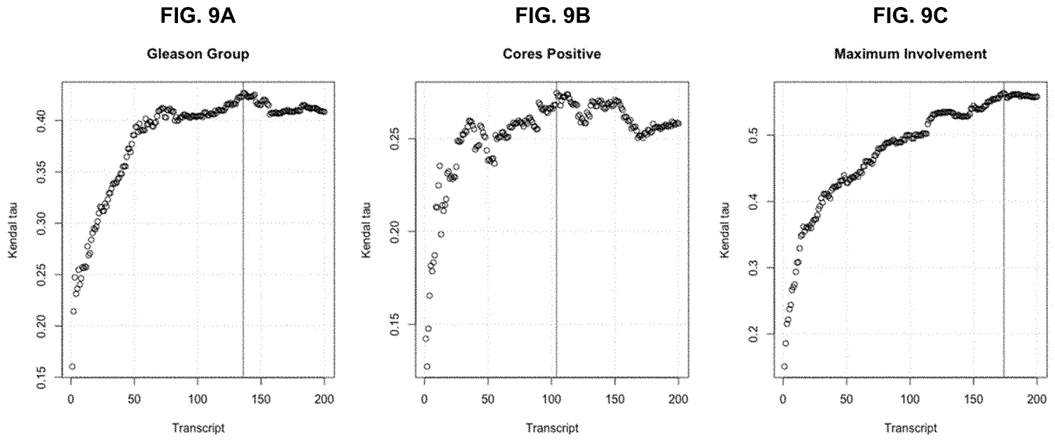

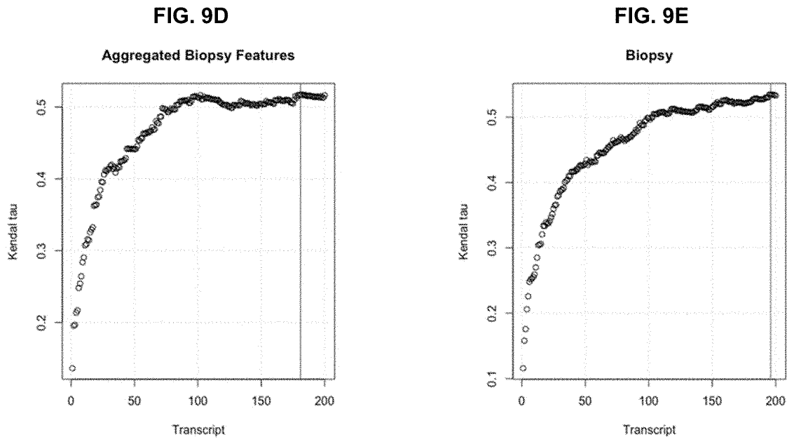

To rank transcripts associated with the summaries of the biopsy being considered, namely, grade group, cores positive, maximum involvement, aggregated biopsy features, and positive vs. negative biopsy, associations between the final set of 10,643 transcripts quantified as log(CD2/CD14) were tested, one transcript at the time via univariate testing. Specifically, a generalized linear model (cumulative logit) was used (McCullagh P., Journal of the Royal Statistical Society Series B (Methodological), 42: 109-42 (1980)), accounting for the ordinal nature of grade group, cores positive, and aggregated biopsy features. Maximum involvement was treated as a continuous endpoint. Greedy optimization was performed for the subset of transcripts that maximizes the association between each endpoint, and a signature score was calculated as the average of the positively associated transcripts minus the negatively associated transcripts. The direction of the association was obtained from the sign of the previously calculated regression coefficients of the generalized linear model used for univariate testing. The agreement between the signature score and the endpoint was then estimated via Kendall's τ-b and the signature score was selected that maximized agreement with the endpoint. Kendall's correlation coefficient (τ-b) was used to account for the fact that the endpoints contained repeated values (grade group, cores positive, and aggressiveness are ordinal, and involvement had considerable repetition) (Kruskal W H, Journal of the American Statistical Association, 53: 814-61 (1958)). FIGS. 9A-9D show results of the procedure for all summaries of the biopsy. Table 4 shows the size of the signature (number of transcripts involved in the averaging), Kendall's τ-tau, the p-value for the hypothesis that endpoint and signature are not correlated (rejecting τ-b=0), and the agreement of the signature with predefined partitions of the endpoints, grade group >1, cores positive >3, maximum involvement >25%, and aggregated biopsy features >1, via Student's t-tests corrected for multiple testing (FDR, Benjamini-Hochberg (50)) (see Table 7). Additionally, signature scores were obtained for other modalities of the transcriptomics data, namely, CD2 only, denoted log(CD2), CD14 only, as log(CD14), and aggregated CD2-CD14 counts, as log(CD2+CD14) (see Table 4). For the cohort characteristics shown in Table 4, univariate testing between the summaries of the biopsy (GG, CP, MI and ABP) and clinical covariates was performed using chi-square tests for discrete variables (ECOG, family history, race and digital rectal exam [DRE]) and t-test for continuous variables (age, log-transformed prostate volume and PSA).

TABLE 4
Gene expression signature characteristics (excluding negative biopsies, N = 340) for clinical
covariates (age, prostate volume and total PSA), and different modalities of the transcriptomics
data, namely, log-transformed ratio, log(CD2/CD14), log-transformed total expression, log(CD2 +
CD14), log-transformed CD2 (phagocytes) and log-transformed CD-14 (lymphocytes). Metrics
correspond to Kendall's τ-b (τ-b), its p-value and the size of the signature (m).

| | Gleason Group | | | Cores Positive | | | Max Involvement |
|---|---|---|---|---|---|---|---|
| Data type | τ-b | p-value | m | τ-b | p-value | m | τ-b |
| log(CD2/CD14) | 0.427 | 1.3 × 10⁻²⁵ | 136 | 0.275 | 3.3 × 10⁻¹¹ | 104 | 0.564 |
| log(CD2 + CD14) | 0.429 | 7.3 × 10⁻²⁶ | 94 | 0.328 | 3.2 × 10⁻¹⁵ | 54 | 0.371 |
| log(CD2) | 0.404 | 3.8 × 10⁻²³ | 198 | 0.284 | 7.4 × 10⁻¹² | 41 | 0.330 |
| log(CD14) | 0.405 | 3.2 × 10⁻²³ | 129 | 0.258 | 4.2 × 10⁻¹⁰ | 188 | 0.544 |
| Age | 0.127 | 2.4 × 10⁻³ | | 0.092 | 3.1 × 10⁻² | | 0.077 |
| Prostate volume | −0.107 | 9.4 × 10⁻³ | | −0.157 | 2.0 × 10⁻⁴ | | −0.166 |
| Total PSA | 0.267 | 9.0 × 10⁻¹¹ | | 0.318 | 4.5 × 10⁻¹⁴ | | 0.252 |

| | Max Involvement | | Aggregated Biopsy Features | | | Biopsy | | |
|---|---|---|---|---|---|---|---|---|
| Data type | p-value | m | τ-b | p-value | m | τ-b | p-value | m |
| log(CD2/CD14) | 8.5 × 10⁻⁴⁴ | 174 | 0.517 | 7.2 × 10⁻³⁷ | 181 | 0.535 | 6 × 10⁻⁶⁷ | 196 |
| log(CD2 + CD14) | 4.4 × 10⁻²⁰ | 159 | 0.373 | 4.0 × 10⁻²⁰ | 133 | 0.510 | 4 × 10⁻⁶¹ | 157 |
| log(CD2) | 2.6 × 10⁻¹⁶ | 184 | 0.317 | 5.3 × 10⁻¹⁵ | 198 | 0.448 | 1 × 10⁻⁴⁷ | 197 |
| log(CD14) | 5.7 × 10⁻⁴¹ | 200 | 0.514 | 2.1 × 10⁻³⁶ | 157 | 0.550 | 9 × 10⁻⁷¹ | 191 |
| Age | 6.2 × 10⁻² | | 0.095 | 2.3 × 10⁻² | | 0.064 | 4 × 10⁻² | |
| Prostate volume | 5.4 × 10⁻⁵ | | −0.197 | 1.8 × 10⁻⁵ | | −0.213 | 8 × 10⁻¹² | |
| Total PSA | 7.7 × 10⁻¹⁰ | | 0.265 | 1.2 × 10⁻¹⁰ | | 0.157 | 4 × 10⁻⁷ | |

TABLE 5
Cohort characteristics.

| | | | | Gleason | Cores | Max | Aggregated Biopsy |
|---|---|---|---|---|---|---|---|
| Covariate | Summary Statistics | | | Grade | Positive | Involvement | Features |
| ECOG | 0 | 675 | (94.7%) | 8.37 × 10⁻⁰³ | 3.83 × 10⁻⁰¹ | 1.85 × 10⁻⁰¹ | 1.11 × 10⁻⁰¹ |
| | 1 | 38 | (5.3%) | | | | |
| Family History | Unknown | 48 | (6.7%) | 2.84 × 10⁻⁰¹ | 1.11 × 10⁻⁰² | 2.01 × 10⁻⁰¹ | 2.86 × 10⁻⁰² |
| | None | 494 | (69.3%) | | | | |
| | Any | 171 | (24.0%) | | | | |
| Race | African American | 69 | (9.7%) | 1.03 × 10⁻⁰¹ | 8.97 × 10⁻⁰¹ | 5.10 × 10⁻⁰¹ | 5.61 × 10⁻⁰¹ |
| | White | 598 | (83.9%) | | | | |
| | Hispanic | 10 | (1.4%) | | | | |
| | Other/Unknown | 36 | (5.1%) | | | | |
| DRE | Unknown | 49 | (6.9%) | 4.05 × 10⁻⁰³ | 1.28 × 10⁻⁰² | 1.73 × 10⁻⁰⁶ | 5.68 × 10⁻⁰⁶ |
| | Negative | 550 | (77.1%) | | | | |
| | Positive | 114 | (16.0%) | | | | |

| | | | | | |
|---|---|---|---|---|---|
| Age | 65 (IQR: 59-69; min: 40; max: 86)* | 1.86 × 10⁻⁰⁴ | 8.21 × 10⁻⁰⁴ | 5.31 × 10⁻⁰⁴ | 4.97 × 10⁻⁰⁴ |
| Prostate volume | 40.8 (IQR: 31.2-55.9; min: 9.5; max: 309) | 7.13 × 10⁻¹³ | 1.64 × 10⁻¹⁴ | 6.45 × 10⁻¹⁵ | 3.78 × 10⁻¹⁵ |
| Total PSA | 5.1 (IQR: 3.8-7.3; min: 0.2; max: 210.7) | 1.20 × 10⁻²¹ | 3.06 × 10⁻²² | 4.94 × 10⁻¹⁸ | 4.20 × 10⁻¹⁷ |

*IQR: interquartile range.

Results

Cohort characteristics.

Prostate volume, age, DRE, race, family history, Gleason grade group, and serum total PSA levels (determined prior to biopsy) are shown in fable 4 (additional details of the biopsy features are in Table 6). Statistically significant associations were observed between prostate volume, total PSA, and all biopsy features (GG, CP, MI and ABP) and modest (borderline) associations for DRE and age.

TABLE 6
Cohort Characteristics.

| | | | Aggregated | | |
|---|---|---|---|---|---|
| | | | Biopsy | | |
| Gleason Group | Count | Percentage | Features | Count | Percentage |
| 0 | 366 | 51.33 | 0 | 366 | 51.33 |
| 1 | 105 | 14.73 | 1 | 71 | 9.96 |
| 2 | 139 | 19.50 | 2 | 51 | 7.15 |
| 3 | 59 | 8.27 | 3 | 78 | 10.94 |
| 4 | 44 | 6.17 | 4 | 147 | 20.62 |
| | | | Max | | |
| Cores Positive | Count | Percentage | Involvement | Count | Percentage |
| 0 | 366 | 51.33 | 0 | 366 | 51.33 |
| 1 | 86 | 12.06 | 10 | 51 | 7.15 |
| 2 | 62 | 8.7 | 20 | 44 | 6.17 |
| 3 | 37 | 5.19 | 30 | 38 | 5.33 |
| 4 | 40 | 5.61 | 40 | 32 | 4.49 |
| 5 | 38 | 5.33 | 50 | 20 | 2.81 |
| 6 | 28 | 3.93 | 60 | 23 | 3.23 |
| 7 | 17 | 2.38 | 70 | 39 | 5.47 |
| 8 | 12 | 1.68 | 80 | 25 | 3.51 |
| 9 | 8 | 1.12 | 90 | 22 | 3.09 |
| 10 | 4 | 0.56 | 100 | 53 | 7.43 |
| 11 | 4 | 0.56 | | | |
| 12 | 11 | 1.54 | | | |

Gene expression signatures of aggressiveness.

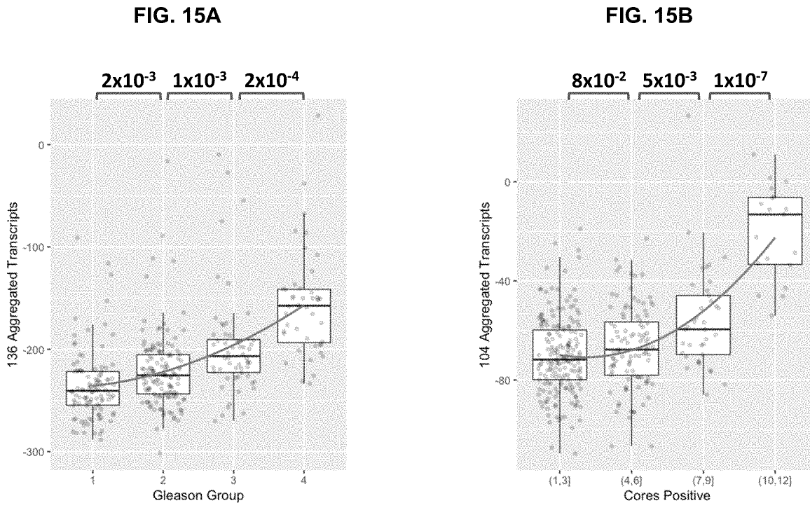

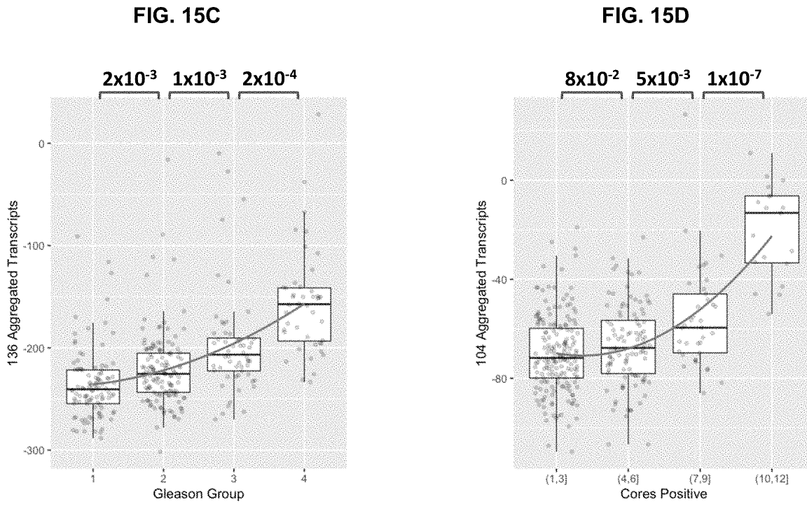

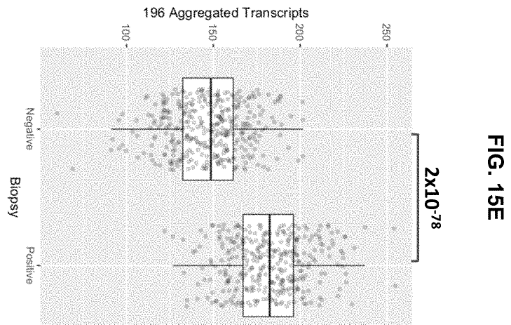

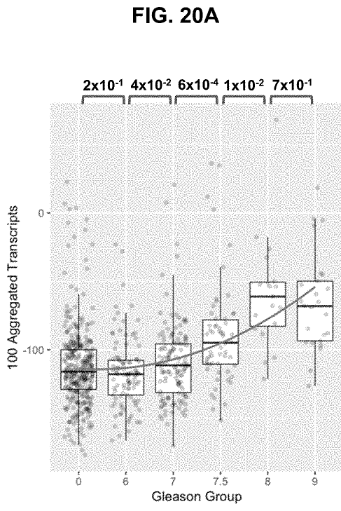

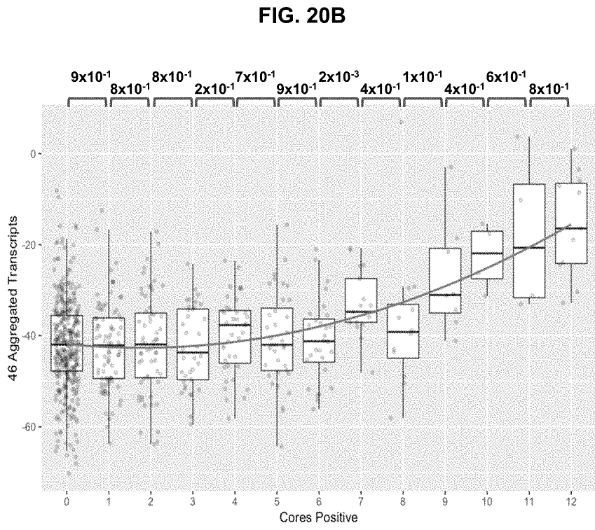

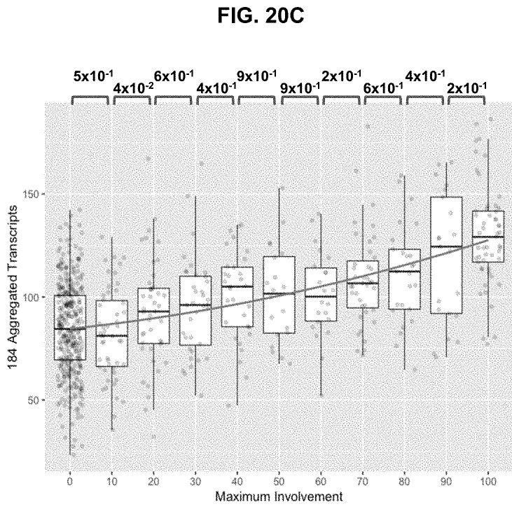

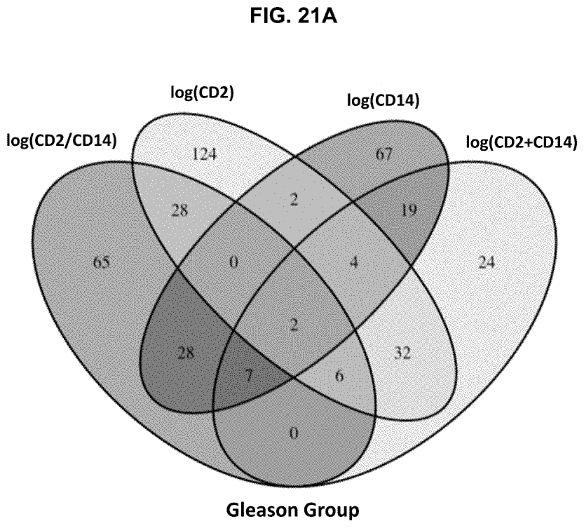

Using normalized gene expression ratios quantified between CD2 and CD14 cells [log(CD2/CD14)] in N=340 positive biopsy subjects obtained via RNA-seq (see RNA sequencing Section), signatures of grade group, cores positive, maximum percent involvement, and aggregated biopsy features were identified. The negative vs. positive biopsy comparison of the complete set of samples (N=713) also was considered. These signatures consisted of m=136, 104, 174, 181 and 196 transcripts (gene symbols), respectively, obtained via greedy optimization (see Statistical Methods). The signature scores shown in FIGS. 15A-15E, calculated as the average of m gene expression ratios (in log domain) while accounting for the direction (sign of the linear regression coefficient) of the association with the endpoints, were significantly associated with grade group (Kendall's τ-b=0.427, p=1.3×10⁻²⁵), cores positive (τ-b=0275, p=3.3×10⁻¹¹), maximum involvement (τ-b=0.564, p=8.5×10⁻⁴⁴), aggregated biopsy features (τ-b=0.517, p=7.2×10⁻³⁷) and negative vs. positive biopsies (τ-b=0.535, p=5.7×10⁻⁶⁷). FIGS. 15A-15E show associations between adjacent subgroups for each biopsy feature, suggesting that most were statistically significant. For instance, consistent, gradual, and significant associations (α=0.05 level) were observed for all adjacent subgroups in grade group, maximum involvement, aggregated biopsy features, and negative vs. positive biopsy. In the case of cores positive, the differences between [1,3] and [4,6] positive cores were not significant (p=0.08), but the other two comparisons were significant, especially [7,9] vs. [10,12] cores (p=1×10⁻⁷). Note that in FIGS. 15A-15E, endpoint values were grouped for grade group [3 and 4], cores positive [1-3, 4-6, 7-9, and 10-12], and maximum involvement [0-25, 25-50, 50-75 and 75-100] to highlight the association with the signature scores; however, FIGS. 20A-20C show signature scores on the full range of the endpoints, which confirmed the trends seen in FIGS. 15A-15E.

The data shown in FIGS. 15A-15E support that (i) negative biopsies have gene signature variation that is larger than that of any of the other groups in all endpoints, and (ii) there is a substantial association between gene expression, quantified as ratios between CD2 and CD14, and aggressiveness in prostate cancer. Substantially more variation in prostate cancer heterogeneity was observed as compared to other groups in positive biopsy strata, especially in negative biopsies.

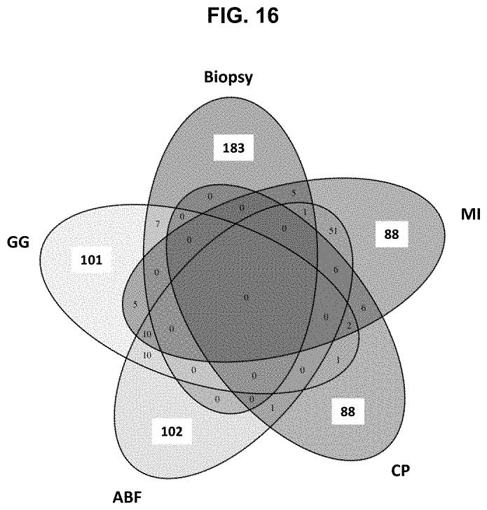

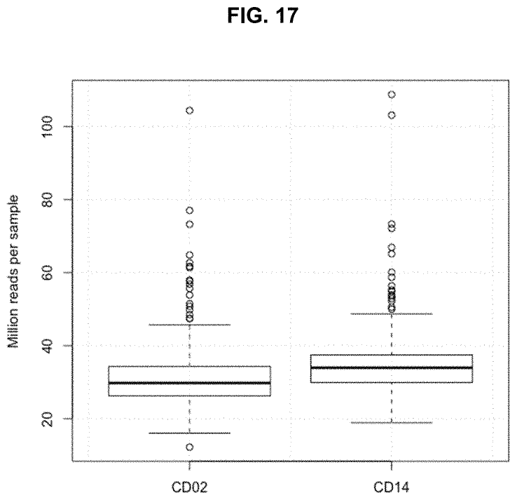

Examining the content of the gene signatures described above revealed that their overall overlap is null (see FIGS. 16A-16E). However, pairwise overlaps were substantial (e.g., 17 transcripts between grade group and maximum involvement, 14 transcripts between cores positive and maximum involvement, and 3 transcripts between grade group and cores positive). As expected, the aggregated biopsy features, being an aggregate of the other three, naturally overlapped: 20 transcripts with grade group, 7 with cores positive, and 68 with maximum involvement. Also, the signature for negative vs. positive biopsy shared 13 transcripts with the other positive-biopsy-exclusive signatures. Interestingly, there were transcripts specific to each endpoint, even for aggregated biopsy features: 108 for grade group, 88 for cores positive, 93 for maximum involvement, 102 for aggregated biopsy features, and 183 for negative vs. positive biopsy. These results highlight the complementary nature of the endpoints, including the aggregated biopsy features introduced here.

Other modalities of the obtained gene expression data were explored, namely CD2 only, CD14 only, and aggregated CD2 and CD14 data, the latter as a proxy for peripheral blood mononuclear cell (PBMC) content. These were denoted as log(CD2), log(CD14), and log(CD2+CD14). Table 4 shows quantitative metrics (tau and p-value) and signature sizes (m) for all modalities and endpoints. For Gleason grade, positive cores, maximum involvement, aggressiveness index, and negative vs. positive biopsy, log(CD2+CD14) (τ-b=0.429, p=′7.3×10⁻²⁶), log(CD2+CD14) (τ-b=0.328, p=3.2×10⁻¹⁵), log(CD2/CD14) (τ-b=0.564, p=8.5×10⁻⁴⁴), log(CD2/CD14) (τ-b=0.517, p=′7.2×10⁻³⁷) and log(CD14) (τ-b=0.550, p=9.0×10⁻⁷¹), respectively, exhibited the highest agreement with each endpoint.

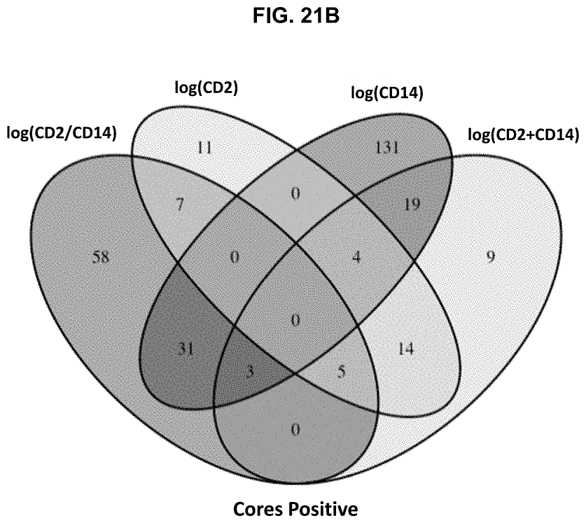

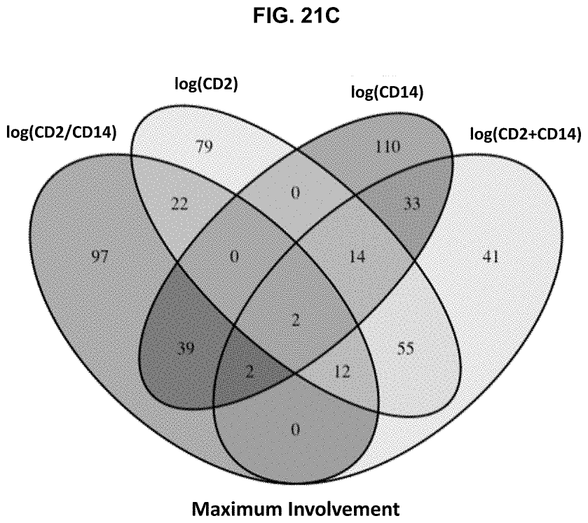

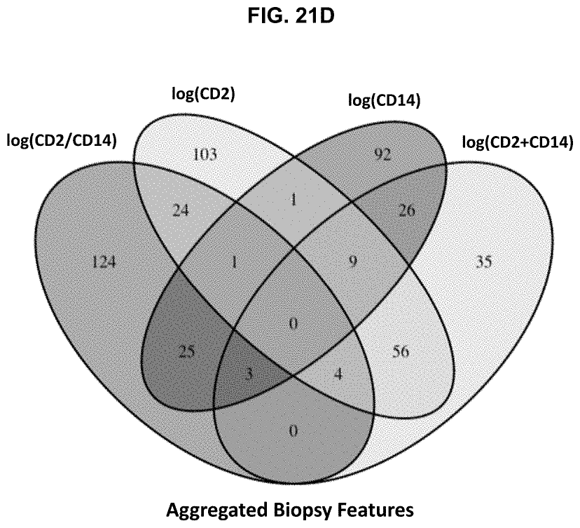

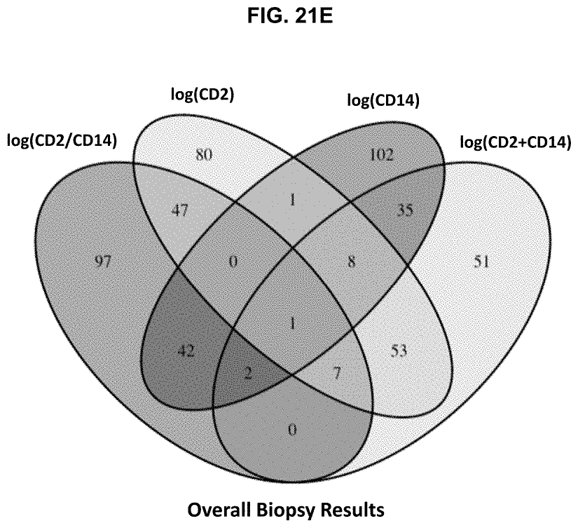

These results suggest that individual components of the assay, CD2 and CD14, are strongly associated with different summaries of the biopsy. In addition, when aggregated into a single aggregated biopsy feature, the ratio, which quantifies relative changes between CD2 and CD14 cells, performs the best in terms of the strength of the association. For context, in Table 4 quantitative metrics are shown for other well-known predictors of aggressiveness in prostate cancer, namely, age, prostate volume, and total PSA, and shows that gene expression signature scores were observably better for grade group, maximum involvement, and aggregated biopsy features. For the case of cores positive, total PSA had the strongest association (τ-b=0.318, p=4.5×10⁻¹⁴), which is the closest to log(CD2+CD14) from all the clinical features. This suggests that gene expression and other predictors may have complementary information that could be leveraged for improved associations with aggressiveness in prostate cancer. Similar to the data shown in FIG. 16, the overlap between the signatures from different modalities was examined for each biopsy feature (see FIGS. 21A-21E) and it was found that there was a high degree of specificity of the transcripts that are informative for each modality.

Summaries of biopsies are often interpreted as dichotomous in clinical practice. For this scenario, the agreement between all signature scores and Gleason grade group >1, cores positive >3, maximum involvement >25% and aggregated biopsy features >1 were estimated via univariate testing (false discovery corrected Student's t-tests). Table 7 shows that for grade group, cores positive, maximum involvement, aggregated biopsy features, and negative vs. positive biopsy, the best gene expression predictors were log(CD2+CD14) (p=5×10⁻¹⁵), log(CD2+CD14) (p=4×10⁻¹¹), log(CD2/CD14) (p=2×10⁻³²) and log(CD2/CD14) (p=2×10⁻²⁹). Except for cases of cores positive where total PSA was the best predictor by a small margin (p=2×10⁻¹¹), gene expression data was a better predictor than age, prostate volume, and total PSA.

TABLE 7
Statistical significance of gene signatures and clinical covariates with
respect to Gleason group >1, aggregated biopsy features >1, cores
positive >3 and maximum involvement >25%. Significance of group
differences quantified via Student's t-tests.

| | | | | Aggregated |
|---|---|---|---|---|
| | Gleason | Cores | Max | Biopsy |
| Data type | Group | Positive | Involvement | Features |
| log (CD2/CD14) | 6 × 10⁻¹¹ | 4 × 10⁻⁷ | 2 × 10⁻³² | 2 × 10⁻²⁹ |
| log (CD2 + CD14) | 5 × 10⁻¹⁵ | 4 × 10⁻¹¹ | 3 × 10⁻¹⁴ | 1 × 10⁻¹¹ |
| log (CD2) | 2 × 10⁻¹² | 2 × 10⁻⁷ | 9 × 10⁻¹¹ | 7 × 10⁻⁹ |
| log (CD14) | 3 × 10⁻¹⁰ | 6 × 10⁻⁷ | 4 × 10⁻³⁰ | 6 × 10⁻²⁵ |
| Age | 2 × 10⁻² | 5 × 10⁻³ | 6 × 10⁻² | 4 × 10⁻² |
| Prostate volume | 1 × 10⁻⁴ | 6 × 10⁻⁵ | 5 × 10⁻⁵ | 2 × 10⁻⁵ |
| Total PSA | 4 × 10⁻⁹ | 2 × 10⁻¹¹ | 1 × 10⁻⁸ | 6 × 10⁻⁸ |

The results of this Example demonstrate that multiple gene expression profiles identified from CD2 and CD14 RNA sequence analysis correlate with prostate biopsy features known to be associated with aggressive biologic behavior. These include number of positive cores, maximum grade group, and maximum cross-sectional surface area of core involvement by tumor. The information from individual immune cell types along with their mathematical combinations are predictive of these parameters. The CD2/CD14 ratio SNEP approach described herein can be understood as a means of normalizing the expression of genes unrelated to a specific disease and can be instrumental in the development of a genomic signature of prostate cancer aggressiveness. The results strongly suggest that analysis of RNA expression data from the body's immune surveillance cells has the potential to capture and summarize the entire heterogeneous tumor and may be a useful tool for discovery of meaningful signatures in prostate cancer.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

1. A method of measuring a panel of biomarkers in a subject, the method comprising: obtaining a biological sample from the subject; determining a measurement for the panel of biomarkers in the biological sample, wherein the panel of biomarkers comprise five or more biomarkers selected from Table 1, Table 2, and/or Table 3, and wherein the measurement comprises measuring a level of each of the biomarkers in the panel.

2. The method of claim 1, wherein the panel of biomarkers comprise six, seven, eight, nine, ten, eleven, twelve, thirteen, fourteen or more biomarkers selected from Table 1, Table 2, and/or Table 3.

3. (canceled)

4. The method of claim 1, further comprising obtaining one or more clinical data from the subject selected from the group consisting of age, race, digital rectal exam (DRE), prostate volume, total prostate-specific antigen (PSA), tumor stage, tumor grade, tumor size, tumor visual characteristics, tumor growth, tumor thickness, tumor progression, tumor metastasis, tumor distribution within the body, odor, molecular pathology, genomics, and/or tumor angiograms, wherein the one or more clinical data are used as clinical covariates and concatenated with the biomarker levels and input into a sparse rank regression model to generate a prostate cancer aggressiveness index.

5. The method of claim 1, wherein the biological sample comprises CD2+ cells and/or CD14+ cells, and determining a measurement for the panel of biomarkers in the biological sample comprises measuring a level of each of the biomarkers in the panel in CD2+ cells and/or CD14+ cells.

6. (canceled)

7. A method of measuring a panel of biomarkers in a subject, the method comprising: obtaining a biological sample from the subject; determining a measurement for the panel of biomarkers in the biological sample, wherein the panel of biomarkers comprise five or more biomarkers selected from of C11orf94, C9orf135, DSP, EGFL6, FST, FSTL1, GATA2, GRID1, KLF17, KRTAP5-8, MID1, MYO1D, OOEP, RSPH9 and TAGLN3, and wherein the measurement comprises measuring a level of each of the biomarkers in the panel.

8.-9. (canceled)

10. The method of claim 7, wherein the panel of biomarkers comprise C11orf94, C9orf135, DSP, EGFL6, FST, FSTL1, GATA2, GRID1, KLF17, KRTAP5-8, MID1, MYO1D, OOEP, RSPH9 and TAGLN3.

11. The method of claim 7, wherein the biological sample comprises CD2+ cells and/or CD14+ cells, and determining a measurement for the panel of biomarkers in the biological sample comprises measuring a level of each of the biomarkers in the panel in CD2+ cells and/or CD14+ cells.

12. (canceled)

13. The method according to claim 7, further comprising obtaining one or more clinical data from the subject selected from the group consisting of age, race, digital rectal exam (DRE), prostate volume, and total prostate-specific antigen (PSA).

14. (canceled)

15. The method of claim 7, wherein measuring a level of each of the biomarkers in the panel comprises measuring gene expression levels or protein expression levels.

16.-21. (canceled)

22. The method of claim 7, wherein the subject is a human.

23. The method of claim 7, further comprising identifying the subject's prostate cancer aggressiveness index value.

24. A kit for performing the measurement of the panel of biomarkers of the subject in claim 1, wherein the kit comprises reagents for measuring at least two of the panel of biomarkers.

25.-31. (canceled)

32. A method for identifying a compound capable of ameliorating or treating prostate cancer in a subject comprising:
a) measuring the levels of two or more markers selected from Table 1, Table 2, and/or Table 3 in a population of the subject's macrophages, monocytes, and/or neutrophils before administering the compound to the subject;
b) measuring the levels of the one or more selected markers in a population of the subject's non-phagocytic cells before administering the compound to the subject;
c) identifying a first difference between the measured levels of the one or more selected markers in steps a) and b);
d) measuring the levels of the one or more selected markers in a population of the subject's macrophage or monocyte cells after the administration of the compound;
e) measuring the levels of the one or more selected markers in a population of the subject's non-phagocytic cells after the administration of the compound;
f) identifying a second difference between the measured levels of the one or more selected markers in steps d) and e); and
g) identifying a difference between the first difference and the second difference, wherein the difference identified in g) indicates that the compound is capable of ameliorating or treating said prostate cancer in the subject.

33. The method of claim 32, further comprising measuring at least one standard parameter associated with said prostate cancer selected from tumor stage, tumor grade, tumor size, tumor visual characteristics, tumor growth, tumor thickness, tumor progression, tumor metastasis tumor distribution within the body, odor, molecular pathology, genomics, and tumor angiograms.

34. (canceled)

35. The method of claim 32, wherein the selected markers are measured from the same or different population of non-phagocytic cells in steps b) or e).

36. (canceled)

37. The method of claim 32, wherein at least two, three, four, five, six, seven, eight, nine, ten, eleven, twelve, thirteen, fourteen, or fifteen markers are selected.

38.-40. (canceled)

41. The method of claim 32, wherein the macrophages, monocytes, and/or neutrophils are isolated from a bodily fluid sample, tissues, or cells of the subject.

42. The method of claim 32, wherein the non-phagocytic cells are isolated from a bodily fluid sample, tissues, or cells of the subject.

43. (canceled)

44. The method of claim 32, wherein the measured levels are gene expression levels or protein expression levels.

45.-50. (canceled)

51. The method of claim 32, wherein the subject is a human or a mammal other than an human.

52.-54. (canceled)
